(19) United States
(12) Patent Application Publication
(10) Pub. No.: US 2005/0272915 A1
(43) Pub. Date: Dec. 8, 2005

(54) Skrp, astray, string, vacm associated with metabolic control

(76) Inventors: Karsten Eulenberg, Bovenden (DE); Gunter Bronner, Gottingen (DE)

Correspondence Address: SUTHERLAND ASBILL & BRENNAN LLP, 999 PEACHTREE STREET, N.E., ATLANTA, GA, 30309, US

(21) Appl. No.: 10/529,366

(22) PCT Filed: Sep. 29, 2003
(86) PCT No.: PCT/EP03/10799

§ 371 Date: Mar. 28, 2005

(30) Foreign Application Priority Data
Sep. 27, 2002 (EP) 02021916.8

(51) Int. Cl. C12Q 1/68; C07H 21/04; A61K 38/17; A61K 48/00; C07K 14/82

(52) U.S. Cl. 530/350; 536/23.5; 435/69.1; 435/6; 435/320.1; 435/325; 514/12; 514/44

(57) ABSTRACT

The present invention discloses novel uses for energy homeostasis regulating proteins and polynucleotides encoding these in the diagnosis, study, prevention, and treatment of metabolic diseases and disorders.

This invention relates to the use of CG7042 (Gadfly Accession Number), astray (GadFly Accession Number CG3705), string (GadFly Accession Number CG1395), or CG1401 (GadFly Accession Number) homologous proteins, to the use of polynucleotides encoding these, and to the use of effectors/modulators of the proteins and polynucleotides in the diagnosis, study, prevention, and treatment of obesity or/and diabetes or/and metabolic syndrome.

There are several metabolic diseases of human and animal metabolism, e.g., obesity and severe weight loss, that relate to energy imbalance where caloric intake versus energy expenditure is imbalanced. Obesity is one of the most prevalent metabolic disorders in the world. It is still a poorly understood human disease that becomes as a major health problem more and more relevant for western society. Obesity is defined as a body weight more than 20% in excess of the ideal body weight, frequently resulting in a significant impairment of health. It is associated with an increased risk for cardiovascular disease, hypertension, diabetes, hyperlipidaemia and an increased mortality rate. Besides severe risks of illness, individuals suffering from obesity are often isolated socially.

Obesity is influenced by genetic, metabolic, biochemical, psychological, and behavioral factors, and can be caused by different reasons such as non-insulin dependent diabetes, increase in triglycerides, increase in carbohydrate bound energy and low energy expenditure. As such, it is a complex disorder that must be addressed on several fronts to achieve lasting positive clinical outcome.

Since obesity is not to be considered as a single disorder but as a heterogeneous group of conditions With (potential) multiple causes, it is also characterized by elevated fasting plasma insulin and an exaggerated insulin response to oral glucose intake (Koltermann O. G., (1980) J. Clin. Invest 65: 1272-1284). A clear involvement of obesity in type 2 diabetes mellitus can be confirmed (Kopelman P. G., (2000) Nature 404: 635-643).

Hyperlipidemla and elevation of free fatty acids correlate clearly with the metabolic syndrome, which is defined as the linkage between several diseases, including obesity and insulin resistance. This often occurs in the same patients and are major risk factors for development of type 2 diabetes and cardiovascular disease. It was suggested that the control of lipid levels and glucose levels is required to treat type 2 diabetes, heart disease, and other occurances of metabolic syndrome (see, for example, Santomauro A. T. et al., (1999) Diabetes, 48: 1836-1841 and Lakka H. M. et al., (2002) JAMA 288: 2709-2716).

Diabetes is a very disabling disease, because medications do not control blood sugar levels well enough to prevent swinging between high and low blood sugar levels. Patients with diabetes are at risk for major complications, including diabetic ketoacidosis, end-stage renal disease, diabetic retinopathy and amputation. There are also a host of related conditions, such as metabolic syndrome, obesity, hypertension, heart disease, peripheral vascular disease, and infections, for which persons with diabetes are at substantially increased risk. The treatment of these complications contributes to a considerable degree to the enormous cost which is imposed by diabetes on health care systems world wide.

The concept of ‘metabolic syndrome’ (syndrome x, insulin-resistance syndrome, deadly quartet) was first described 1966 by Camus and reintroduced 1988 by Reaven (Camus J. P., (1966) Rev Rhum Mal Osteoartic 33: 10-14; Reaven G. M. et al., (1988) Diabetes, 37: 1595-1607). Today metabolic syndrome is commonly defined as clustering of cardiovascular risk factors like hypertension, abdominal obesity, high blood levels of triglycerides and fasting glucose as well as low blood levels of HDL cholesterol. Insulin resistance greatly increases the risk of developing the metabolic syndrome (Reaven G., (2002) Circulation 106: 286-288). The metabolic syndrome often precedes the development of type II diabetes and cardiovascular disease (Lakka H. M. et al., 2002, supra).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms or/and molecules influencing obesity or body weight/body mass regulations are not known. In addition, several single-gene mutations resulting in obesity have been described in mice, implicating genetic factors in the etiology of obesity (Friedman J. M. and Leibel R. L., (1992), Cell 69: 217-220). In the obese (ob) mouse a single gene mutation (obese) results in profound obesity, which is accompanied by diabetes (Friedman J. M. et. al., (1991) Genomics 11: 1054-1062).

Therefore, the technical problem underlying the present invention was to provide for means and methods for modulating (pathological) metabolic conditions influencing body-weight regulation or/and energy homeostatic circuits. The solution to said technical problem is achieved by providing the embodiments characterized in the claims.

Accordingly, the present invention relates to novel functions of proteins and nucleic acids encoding these in body-weight regulation, energy homeostasis, metabolism, and obesity. Further new compositions are provided that are useful in diagnosis, treatment, and prognosis of metabolic diseases and disorders as described.

So far, it has not been described that a protein of the invention or a homologous protein are involved in the regulation of energy homeostasis and body-weight regulation and related disorders, and thus, no functions in metabolic diseases and dysfunctions and other diseases as listed above have been discussed.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that CG7042 (GadFly Accession Number), astray (GadFly Accession Number CG3705), string (GadFly Accession Number CG1395), or CG1401 (GadFly Accession Number) homologous proteins (herein referred to as “proteins of the invention” or “a protein of the invention”) are regulating the energy homeostasis and fat metabolism, especially the metabolism and storage of triglycerides, and polynucleotides, which identify and encode the proteins disclosed in this invention. The invention also relates to vectors, host cells, and recombinant methods for producing the polypeptides and polynucleotides of the invention. The invention also relates to the use of these compounds and effectors/modulators thereof, e.g. antibodies, biologically active nucleic acids, such as artisense molecules, RNAi molecules or ribozymes, aptamers, peptides or low-molecular weight organic compounds recognizing said polynucleotides or polypeptides, in the diagnosis, study, prevention, and treatment of metabolic diseases or dysfunctions, including metabolic syndrome, obesity, or/and diabetes as well as related disorders such as eating disorder, cachexia, hypertension, coronary heart disease, hypercholesterolemia, dyslipidemia, osteoarthritis, gallstones, or liver fibrosis.

Stress-activated protein kinase (SAPK) pathway-regulating phosphatase 1 (SKRP1) is a member of the mitogen-activated protein kinase (MAPK) phosphatase (MKP) family. SKRP1 interacts physically with the MAPK kinase MKK7, a c-Jun N-terminal kinase (JNK) activator, and inactivates the MAPK JNK pathway. SKRP1 contributes to the precise regulation of JNK signaling and plays a scaffold role for the JNK signaling by selectively forming stable complexes with MKK7 and regulation of the MKK7 activity and MKK7-induced gene transcription (Zama T. et al., (2002) J Biol Chem 277(26): 23919-23926). Mitogen-activated protein kinases (MAPKs) are activated in response to various extracellular stimuli, and their activities are regulated by upstream activating kinases and protein phosphatases such as MAPK phosphatases (MKPs). SKRP1, a member of the MKP family, contains an extended active site sequence motif conserved in all MKPs but lacks a Cdc25 homology domain. SKRP1 interacts with its physiological substrate JNK through MKK7, thereby leading to the precise regulation of JNK activity in vivo (Zama T. et al., (2002) J Biol 277(26): 23909-2391 8).

Another dual specifity protein phosphatase and member of the MKP family, MAPK phosphatase-1 (MKP-1), has been studied in diabetic rats. Protein expression of MKP-1, a dual specificity phosphatase that inactivates MAPK, was decreased in streptozotocin-induced diabetes mellitus (DM) rats. Glomerular MAPK is activated in DM by multiple mechanisms i.e., increases in protein contents, increased phosphorylation, and decreased dephosphorylation of the enzyme due to suppression of MKP-1. These alterations may have an implication in the pathogenesis of diabetic nephropathy (Awazu M. et al., (1 999). J Am Soc Nephrol 10(4):738-745). Gene expression of MKP-1 in hepatectomized liver in type 1 diabetic BB rats is changed (Chin S. et al., (1995) Am J Physiol 269(4 Pt 1): E691-700).

Phosphoserine phosphatase (PSP) is a member of a large class of enzymes that catalyze phosphoester hydrolysis using a phosphoaspartate-enzyme intermediate. PSP is a likely regulator of the steady-state d-serine level in the brain, which is a critical co-agonist of the N-methyl-d-aspartate type of glutamate receptors (Wang W. et al., (2002) J Mol Biol 319(2): 421-431). PSP belongs to a class of phosphotransferases forming an acylphosphate during catalysis (Collet J. F. et al., (1999) J Biol Chem 274(48): 33985-33990).

String is required for mitosis early in development and is transcribed in a dynamic pattern that anticipates the pattern of embryonic cell divisions. Regulated expression of string mRNA controls the timing and location of zygotically driven embryonic cell divisions (Edgar B. A. and O'Farrell P. H., (1989) Cell 57: 177-187; Edgar B. A. and O'Farrell P. H., (1990) Cell 62: 469-480). string regulation is a critical part of the control of early entry into mitosis in some, but not all, G2-arrested imaginal cells. string is essential for the generation of the adult cuticle (Kylsten P. and Saint R., (1997) Dev Biol. 192(2): 509-522). string is required for completion of daughter centriole assembly in embryos (Vidwans S. J. et al., (1 999) J Cell Biol 147(7): 1371-1378).

The Cdc25 family of protein phosphatases positively regulates the cell division cycle by activating cyclin-dependent protein kinases. In humans and rodents, three Cdc25 family members denoted Cdc25A, -B, and -C have been identified. The murine forms of Cdc25 exhibit distinct patterns of expression both during development and in adult mouse tissues. Mice lacking Cdc25C (Cdc25C(−/−) mice) are viable and do not display any obvious abnormalities. Cdc25C is expressed most abundant in testis, followed by thymus, ovary, spleen, and intestine. Cdc25A or/and Cdc25B may compensate for loss of Cdc25C in the mouse (Chen M. S. et al. (2001) Mol Cell Biol 21(12):3853-3861). Cdc25 phosphatases, which dephosphorylate cyclin-dependent kinases, are overexpressed in many human tumors (Pestell K. E. et al., (2000) Oncogene 19(56):6607-661 2).

Vasopressin-activated Ca⁽²⁺⁾-mobilizing (VACM-1), a cullin gene family member, regulates cellular signaling. Overexpression of the VACM-1 receptor results in increased arginine vasopressin (AVP) binding, but does not have amino acid sequence homology with the traditional AVP receptors. VACM-1, however, is homologous with a cullin family of proteins that has been implicated in the regulation of cell cycle through the ubiquitin-mediated degradation of cyclin-dependent kinase inhibitors. The effects of VACM-1 expression on the Ca⁽²⁺⁾ and cAMP-dependent signaling pathway were examined. Expression of the VACM-1 gene reduced cAMP production (Burnatowska-Hledin M. et al., (2000) Am J Physiol Cell Physiol 279(1):C266-273).

So far, it has not been described that the CG7042, astray, string, or CG1401 proteins of the invention or homologous proteins are involved in the regulation of energy homeostasis and body-weight regulation and related disorders, and thus, no functions in metabolic diseases and dysfunctions and other diseases as listed above have been discussed.

CG7042, astray, string, or CG1401 homologous proteins and nucleic acid molecules coding therefore are obtainable from insect or vertebrate species, e.g. mammals or birds. Particularly preferred are homologous nucleic acids, particularly nucleic acids encoding a human protein as described in Table 1.

The invention particularly relates to nucleic acid molecules encoding polypeptides contributing to regulating the energy homeostasis or/and the metabolism of triglycerides, wherein said nucleic acid molecule comprises
(a) the nucleotide sequence encoding Drosophila CG7042, astray, string, or CG1401 or human homologous nucleic acids, particularly nucleic acids encoding a human protein as described in Table 1, or/and a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99,6% identical to the amino acid sequences of the CG7042, astray, string, or CG1401 protein, preferably of the human homologous protein as described in Table 1,
(e) a sequence which differs from the nucleic acid molecule of (a) to (d) by mutation and wherein said mutation causes an alteration, deletion, duplication or/and premature stop in the encoded polypeptide or
(f) a partial sequence of any of the nucleotide sequences of (a) to (e) having a length of at least 15 bases, preferably at least 20 bases, more preferably at least 25 bases and most preferably at least 50 bases, the length being in particular 15-25 bases, preferably 25-35 bases, more preferably 35-50 bases and most preferably at least 50 bases.

The invention is based on the finding that CG7042, astray, string, or CG1401 or/and homologous proteins and the polynucleotides encoding these, are involved in the regulation of triglyceride storage and therefore energy homeostasis. The invention describes the use of these proteins and polynucleotides for the diagnosis, study, prevention, or/and treatment of metabolic diseases or/and dysfunctions, including metabolic syndrome, obesity, or/and diabetes, as well as related disorders such as eating disorder, cachexia, hypertension, coronary heart disease, hypercholesterolemia, dyslipidemia, osteoarthritis, liver fibrosis, or gallstones.

Accordingly, the present invention relates to genes with novel functions in body-weight regulation, energy homeostasis, metabolism, and obesity, functional fragments of said genes, polypeptides encoded by said genes or functional fragments thereof, and, modulators/effectors thereof, e.g. antibodies, biologically active nucleic acids, such as antisense molecules, RNAi molecules, or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease.

A forward genetic screen was performed in fly displaying a mutant phenotype due to misexpression of a known gene (see, St Johnston D., (2002) Nat Rev Genet 3: 176-188; Rorth P., (1996) Proc Natl Acad Sci USA 93: 12418-12422). In this invention, we have used a genetic screen to identify mutations that cause changes in the body weight, which are reflected by a significant change of triglyceride levels.

Obese people mainly show a significant increase in the content of triglycerides. Triglycerides are the most efficient storage for energy in cells. In order to isolate genes with a function in energy homeostasis, several thousand proprietary and publicly available EP-lines were tested for their triglyceride content after a prolonged feeding period (see Examples and Figures for more detail). Lines with significantly changed triglyceride content were selected as positive candidates for further analysis. The increase or decrease of triglyceride content due to the loss or gain of a gene function suggests gene activities in energy homeostasis in a dose dependent manner that controls the amount of energy stored as triglycerides.

The content of triglycerides of a pool of flies with the same genotype was analyzed after prolonged feeding using a triglyceride assay. Male flies homozygous for the integration of vectors for: Drosophila EP-lines were analyzed in an assay measuring the triglyceride contents of these flies, illustrated in more detail in the Examples section. The results of the triglyceride content analysis are shown in FIGS. 1, 4, 8, and 11, respectively.

Genomic DNA sequences were isolated that are localized adjacent to the EP vector integration. Using those isolated genomic sequences public databases like Berkeley Drosophila Genome Project (GadFly; see also FlyBase (1999) Nucleic Acids Research 27:85-88) were screened thereby identifying the integration site of the vectors, and the corresponding genes, described in more detail in the Examples section. The molecular organization of the genes is shown in FIGS. 2, 5, 9, and 12, respectively.

The Drosophila genes and proteins encoded thereby with functions in the regulation of triglyceride metabolism were further analysed in publicly available sequence databases (see Examples for more detail) and mammalian homologs were identified.

The function of the mammalian homologs in energy homeostasis was further validated in this invention by analyzing the expression of the transcripts in different tissues and by analyzing the role in adipocyte differentiation. Expression profiling studies (see Examples for more detail) confirm the particular relevance of the protein(s) of the invention as regulators of energy metabolism in mammals. Further, we show that the proteins of the invention are regulated by fasting and by genetically induced obesity. In this invention, we used mouse models of insulin resistance or/and diabetes, such as mice carrying gene knockouts in the leptin pathway (for example, ob (leptin) or db (leptin receptor) mice) to study the expression of the proteins of the Invention. Such mice develop typical symptoms of diabetes, show hepatic lipid accumulation and frequently have increased plasma lipid levels (see Bruning J. C. et al., (1998) Mol. Cell. 2: 559-569).

Microarrays are analytical tools routinely used in bioanalysis. A microarray has molecules distributed over, and stably associated with, the surface of a solid support. The term “microarray” refers to an arrangement of a plurality of polynucleotides, polypeptides, antibodies, or other chemical compounds on a substrate. Microarrays of polypeptides, polynucleotides, or/and antibodies have been developed and find use in a variety of applications, such as monitoring gene expression, drug discovery, gene sequencing, gene mapping, bacterial identification, and combinatorial chemistry. One area in particular in which microarrays find use is in gene expression analysis (see Example 6). Array technology can be used to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Microarrays may be prepared, used, and analyzed using methods known in the art (see for example, Brennan T. M., (1995) U.S. Pat. No. 5,474,796; Schena M. et al., (1996) Proc. Natl. Acad. Sci. USA 93: 10614-10619; Baldeschwieler et al., (1995) PCT application WO9525116; Shalon T. D. and Brown P. O., (1995) PCT application WO9535505; Heller R. A. et al., (1997) Proc. Natl. Acad. Sci. USA 94: 2150-2155; Heller M. J. and Tu E., (1997) U.S. Pat. No. 5,605,662). Various types of microarrays are well known and thoroughly described in Schena M., ed. (1999); DNA Microarrays: A Practical Approach, Oxford University Press, London.

Oligonucleotides or longer fragments derived from any of the polynucleotides described herein may be used as elements on a microarray. The microarray can be used in transcript imaging techniques, which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents, which are highly effective and display the fewest side effects may be selected for a patient based on his/her pharmacogenomic profile.

As determined by Microarray analysis, phosphoserine phosphatase (PSPH), cell division cycle 25B (CDC25B), and cullin 5 (CUL5) show differential expression in human primary adipocytes. Thus, PSPH, CDC25B, and CUL5 are strong candidates for the manufacture of a pharmaceutical composition and a medicament for the treatment of conditions related to human metabolism, such as obesity, diabetes, or/and metabolic syndrome.

The invention also encompasses polynucleotides that encode the proteins of the invention or homologous proteins. Accordingly, any nucleic acid sequence, which encodes the amino acid sequences of the proteins of the invention or homologous proteins, can be used to generate recombinant molecules that express the proteins of the invention or homologous proteins. In a particular embodiment, the invention encompasses a nucleic acid encoding Drosophila CG7042, astray, string, or CG1401, or human CG7042, astray, string, or CG1401 homologs, preferably a human homologous protein as described in Table 1; referred to herein as the proteins of the invention. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. The invention contemplates each and every possible variation of nucleotide sequence that can be made by selecting combinations based on possible codon choices.

Also encompassed by the invention are polynucleotide sequences that are capable of hybridizing to the claimed nucleotide sequences, and in particular, those of the polynucleotide encoding the proteins of the invention, under various conditions of stringency. Hybridization conditions are based on the melting temperature (Tm) of the nucleic acid binding complex or probe, as taught in Wahl G. M. et al., (1987: Methods Enzymol. 152: 399-407) and Kimmel A. R. (1987; Methods Enzymol. 152: 507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C., and most preferably at 68° C., a positive hybridization signal is observed. Altered nucleic acid sequences encoding the proteins which are encompassed by the invention include deletions, insertions or substitutions of different nucleotides resulting in a polynucleotide that encodes the same or a functionally equivalent protein.

The encoded proteins may also contain deletions, insertions or substitutions of amino acid residues, which produce a silent change and result in functionally equivalent proteins. Deliberate amino acid substitutions may be made on the basis of similarity in polarity, charge, solubility, hydrophobicity, hydrophilicity, or/and the amphipathic nature of the residues as long as the biological activity of the protein is retained. Furthermore, the invention relates to peptide fragments of the proteins or derivatives thereof such as cyclic peptides, retro-inverso peptides of peptide mimetics having a length of at least 4, preferably at least 6 and up to 50 amino acids.

Also included within the scope of the present invention are alleles of the genes encoding the proteins of the invention or homologous proteins. As used herein, an ‘allele’ or ‘allelic sequence’ is an alternative form of the gene, which may result from at least one mutation in the nucleic acid sequence. Alleles may result in altered mRNAs or polypeptides whose structures or function may or may not be altered. Any given gene may have none, one or many allelic forms. Common mutational changes, which give rise to alleles, are generally ascribed to natural deletions, additions or substitutions of nucleotides. Each of these types of changes may occur alone or in combination with the others, one or more times in a given sequence.

The nucleic add sequences encoding the proteins of the invention or homologous proteins may be extended utilizing a partial nucleotide sequence and employing various methods known in the art to detect upstream sequences such as promoters and regulatory elements. For example, one method which may be employed, ‘restriction-site’ PCR, uses universal primers to retrieve unknown sequence adjacent to a known locus (Sarkar G. et al., (1993) PCR Methods Applic. 2: 318-322). Inverse PCR may also be used to amplify or extend sequences using divergent primers based on a known region (Triglia T. et al., (1988) Nucleic Acids Res. 16: 8186). Another method which may be used is capture PCR which involves PCR amplification of DNA fragments adjacent to a known sequence in human and yeast artificial chromosome DNA (Lagerstrom M. et al., (1991) PCR Methods Applic. 1: 111-119). Another method which may be used to retrieve unknown sequences is that of Parker J. D. et al., (1991) Nucleic Acids Res. 19: 3055-3060. Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express a biologically active protein, the nucleotide sequences encoding the proteins or functional equivalents, may be inserted into appropriate expression vectors, i.e., a vector which contains the necessary elements for the transcription and translation of the inserted coding sequence. Methods, which are well known to those skilled in the art, may be used to construct expression vectors containing sequences encoding the proteins and the appropriate transcriptional and translational control elements. These methods include in vitro recombinant DNA techniques, synthetic techniques, and in vivo genetic recombination. Such techniques are described in Sambrook J. et al. (1989) Molecular Cloning, A Laboratory Manual, Cold Spring Harbor Press, Plainview, N.Y. and Ausubel F. M. et al. (1989) Current Protocols in Molecular Biology, John Wiley & Sons, New York, N.Y.

In a further embodiment of the invention, natural, modified or recombinant nucleic acid sequences encoding the proteins of the invention or homologous proteins may be ligated to a heterologous sequence to encode a fusion protein.

A variety of expression vector/host systems may be utilized to contain and express sequences encoding the proteins or fusion proteins. These include, but are not limited to, micro-organisms such as bacteria transformed with recombinant bacteriophage, plasmid or cosmid DNA expression vectors; yeast transformed with yeast expression vectors; insect cell systems infected with virus expression vectors (e.g., baculovirus, adenovirus, adeno-associated virus, lentiverus, retrovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences of the invention in a sample can be detected by DNA-DNA or DNA-RNA hybridization or amplification using probes or portions or fragments of said polynucleotides. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of labeled RNA probes, PCR amplification using a labeled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent or chromogenic agents as well as substrates, co-factors, inhibitors, magnetic particles, and the like.

Host cells transformed with nucleotide sequences encoding a protein of the invention may be cultured under conditions suitable for the expression and recovery of said protein from cell culture. The protein produced by a recombinant cell may be secreted or contained intracellularly depending on the sequence or/and the vector used. As will be understood by those of skill in the art, expression vectors containing polynucleotides, which encode the protein may be designed to contain signal sequences, which direct secretion of the protein through a prokaryotic or eukaryotic cell membrane. Other recombinant constructions may be, used to join sequences encoding the protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and the desired protein may be used to facilitate purification.

Diagnostics and Therapeutics

The data disclosed in this invention show that the nucleic acids and proteins of the invention are useful in diagnostic and therapeutic applications implicated, for example but not limited to, in metabolic disease or dysfunctions, including metabolic syndrome, obesity or/and diabetes, as well as related disorders such as eating disorder, cachexia, hypertension, coronary heart disease, hypercholesterolemia, dyslipidemia, osteoarthritis, gallstones, or liver fibrosis. Hence, diagnostic and therapeutic uses for the proteins of the invention nucleic adds and proteins of the invention are, for example but not limited to, the following: (i) protein therapy, (ii) small molecule drug target, (iii) antibody target (therapeutic, diagnostic, drug targeting/cytotoxic antibody), (iv) diagnostic or/and prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention and effectors thereof are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acids of the invention or fragments thereof, may further be useful in diagnostic applications, wherein the presence or amount of the nucleic acids or the proteins are to be assessed. Further antibodies that bind immunospecifically to the novel substances of the invention may be used in therapeutic or diagnostic methods.

For example, in one aspect, antibodies, which are specific for a protein of the invention or a homologous protein, may be used directly as a modulator/effector, e.g. an antagonist or indirectly as a targeting or delivery mechanism for bringing a pharmaceutical agent to cells or tissue which express the protein. The antibodies may be generated using methods that are well known in the art. Such antibodies may include, but are not limited to, polyconal, monoclonal, chimeric single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof which has immunogenic properties. Depending on the host species, various adjuvants may be used to increase immunological response. It is preferred that the peptides, fragments or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Kohler G. and Milstein C., (1975) Nature 256: 495-497; Kozbor D. et al. (1985) J. Immunol. Methods 81: 31-42; Cote R. J. et al., (1983) Proc. Natl. Acad. Sci. 80: 2026-2030; Cole S. P. et al., (1984) Mol. Cell Biochem. 62: 109-120).

In addition, techniques developed for the production of ‘chimeric antibodies’, the splicing of mouse antibody genes to human antibody genes to obtain a molecule with appropriate antigen specificity and biological activity can be used (Morrison S. L. et al., (1984) Proc. Natl. Acad. Sci. 81: 6851-6855; Neuberger M. S. et al., (1984) Nature 312: 604-608; Takeda S. et al. (1985) Nature 314:

452-454). Alternatively, techniques described for the production of single chain antibodies may be adapted, using methods known in the art, to produce single chain antibodies specific for the proteins of the invention or homologous proteins. Antibodies with related specificity, but of distinct idiotypic composition, may be generated by chain shuffling from random combinatorial immunoglobulin libraries (Kang A. S. et al., (1991) Proc. Natl. Acad. Sci. 88: 11120-11123). Antibodies may also be produced by inducing in vivo production in the lymphocyte population or by screening recombinant immunoglobulin libraries or panels of highly specific binding reagents as disclosed in the literature (Orlandi R. et al., (1989) Proc. Natl. Acad. Sci. 86: 3833-3837; Winter, G. and Milstein C., (1991) Nature 349: 293-299).

Antibody fragments which contain specific binding sites for the proteins may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse W. D. et al., (1989) Science 246: 1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reacive to two non-interfering protein epitopes are preferred, but a competitive binding assay may also be employed (Maddox D. E. et al., (1 983) J. Exp. Med. 1 58:1211-1216).

In another embodiment of the invention, the polynucleotides of the invention or fragments thereof or nucleic acid modulator/effector molecules such as aptamers, antisense molecules, RNAi molecules, or ribozymes may be used for therapeutic purposes. In one aspect, aptamers, i.e. nucleic acid molecules, which are capable of binding to a protein of the invention and modulating its activity, may be generated by a screening and selection procedure involving the use of combinatorial nucleic acid libraries.

In a further aspect, antisense molecules may be used in situations in which it would be desirable to block the transcription of the mRNA. In particular, cells may be transformed with sequences complementary to polynucleotides encoding the proteins of the invention or homologous proteins. Thus, antisense molecules may be used to modulate protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the proteins of the invention or homologous proteins. These techniques are described both in Sambrook et al. (supra) and in Ausubel et al. (supra). Genes encoding the proteins of the invention or homologous proteins can be turned off by transforming a cell or tissue with expression vectors, which express high levels of polynucleotides that encode the proteins of the invention or homologous proteins or fragments thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing artisense molecules, e.g. DNA, RNA or PNA, to the control regions of the genes encoding the proteins of the invention or homologous proteins, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the proteins of the invention or homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage she may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ or/and 3′ ends of the molecule or modifications in the nucleobase, sugar or/and phosphate moieties, e.g. the use of phosphorothioate or 2O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of the nucleic acids and the proteins of the invention or homologous nucleic acids or proteins, antibodies to the proteins of the invention or homologous proteins, mimetics, agonists, antagonists or inhibitors of the proteins of the invention or homologous proteins or nucleic acids. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination, with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, for example the nucleic acids or the proteins of the invention or homologous proteins or nucleic acids or fragments thereof, antibodies of the proteins of the invention or homologous proteins, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to the proteins may be used for the diagnosis of conditions or diseases characterized by or associated with over- or underexpression of the proteins of the invention or homologous proteins or in assays to monitor patients being treated with the proteins of the invention or homologous proteins, or effectors thereof, e.g. agonists, antagonists, or inhibitors. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FAGS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometry, means. Quantities of protein expressed in control and disease, samples from biopsied tissues are compared with the standard values. Deviation between standard and subject values establishes the parameters for diagnosing disease.

In another embodiment of the invention, the polynucleotides specific for the proteins of the invention or homologous proteins may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with probes which are capable of detecting polynucleotide sequences, including genomic sequences, encoding the proteins of the invention or homologous proteins or closely related molecules, may be used to identify nucleic acid sequences which encode the respective protein. The hybridization probes of the subject invention may be DNA or RNA and derived from the nucleotide sequence of the polynucleotide encoding the proteins of the invention or from a genomic sequence including promoter, enhancer elements, and introns of the naturally occurring gene. Hybridization probes may be labeled by a variety of reporter groups, for example, radionuclides such as ³²P or ³⁵S or enzymatic labels, such as alkaline phosphatase coupled to the probe via avidin/biotin coupling systems, and the like.

Polynucleotide sequences specific for the proteins of the invention or homologous nucleic acids may be used for the diagnosis of conditions or diseases, which are associated with the expression of the proteins. Examples of such conditions or diseases include, but are not limited to, metabolic diseases and disorders, including obesity or/and diabetes. Polynucleotide sequences specific for the proteins of the invention or homologous proteins may also be used to monitor the progress of patients receiving treatment for metabolic diseases and disorders, including obesity or/and diabetes. The polynucleotide sequences may be used qualitative or quantitative assays, e.g. in Southern or Northern analysis, dot blot or other membrane-based technologies; in PCR technologies; or in dip stick, pin, ELISA or chip assays utilizing fluids or tissues from patient biopsies to detect altered gene expression.

In a particular aspect, the nucleotide sequences specific for the proteins of the invention or homologous nucleic acids may be useful in assays that detect activation or induction of various metabolic diseases or dysfunctions, including metabolic syndrome, obesity or/and diabetes, as well as related disorders such as eating disorder, cachexia, hypertension, coronary heart disease, hypercholesterolemia, dyslipidemia, osteoarthritis, gallstones, or liver fibrosis. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable have hybridized with nucleotide sequences in the sample, the presence of altered levels of nucleotide sequences encoding the proteins of the invention or homologous proteins in the sample indicates the presence of the associated disease. Such assays may also be used to evaluate the efficacy of a particular therapeutic treatment regimen in animal studies, in clinical trials or in monitoring the treatment of an individual patient.

In order to provide a basis for the diagnosis of a disease associated with expression of the proteins of the invention or homologous proteins, a normal or standard profile for expression is established. This may be accomplished by combining body fluids or cell extracts taken from normal subjects, either animal or human, with a sequence or a fragment thereof, which is specific for the nucleic acids encoding the proteins of the invention or homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as described above the presence of an unusual amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders.

Additional diagnostic uses for oligonucleotides designed from the sequences encoding the proteins of the invention or homologous proteins may involve the use of PCR. Such oligomers may be chemically synthesized, generated enzymatically or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers or even a degenerate pool of oligomers may be employed under less stringent conditions for detection or/and quantification of closely related DNA or RNA sequences.

In another embodiment of the invention, the nucleic acid sequences may also be used to generate hybridization probes, which are useful for mapping the naturally occurring genomic sequence. The sequences may be mapped to a particular chromosome or to a specific region of the chromosome using well known techniques. Such techniques include FISH, FACS or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.). The results may be correlated with other physical chromosome mapping techniques and genetic map data Examples of genetic map data can be found in the 1994 Genome Issue of Science (265:1981f). Correlation between the location of the gene encoding the proteins of the invention on a physical chromosomal map and a specific disease or predisposition to a specific disease, may help to delimit the region of DNA associated with that genetic disease.

The nucleotide sequences of the subject invention may be used to detect differences in gene sequences between normal, carrier or affected individuals. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the proteins of the invention, their catalytic or immunogenic fragments or oligopeptides thereof, an in vitro model, a genetically altered cell or animal, can be used for screening libraries of compounds, e.g. peptides or low molecular weight organic compounds, in any of a variety of drug screening techniques. One can identify modulators/effectors, e.g. receptors, enzymes, proteins, ligands, or substrates that bind to, modulate or mimic the action of one or more of the proteins of the invention. The protein or fragment thereof employed in such screening may be free in solution, affixed to a solid support, borne on a cell surface, or located intracellularly. The formation of binding complexes, between the protein and the agent tested, may be measured. Agents could also, either directly or indirectly, influence the activity of the proteins of the invention.

In vivo, the enzymatic phosphatase activity of the unmodified polypeptides of the CG7042, astray, or string homologous phosphatases towards a substrate can be measured. Activation of the phosphatase may be induced in the natural context by extracellular or intracellular stimuli, such as signaling molecules or environmental influences. One may generate a system containing a phosphatase, may it be an organism, a tissue, a culture of cells or cell-free environment, by exogenously applying this stimulus or by mimicking this stimulus by a variety of the techniques, some of them described further below. A system containing activated phosphatase may be produced (i) for the purpose of diagnosis, study, prevention, and treatment of diseases and disorders related to body-weight regulation and thermogenesis, for example, but not limited to, metabolic diseases, (ii) for the purpose of identifying or validating therapeutic candidate agents, pharmaceuticals or drugs that influence the genes of the invention or their encoded polypeptides, (iii) for the purpose of generating cell lysates containing activated polypeptides encoded by the genes of the invention, (iv) for the purpose of isolating from this source activated polypeptides encoded by the genes of the invention.

In addition activity of CG7042, astray, or string homologous proteins against their physiological substrate(s) or derivatives thereof could be measured in cell-based assays. Agents may also interfere with posttranslational modifications of the proteins of the invention, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the proteins of the invention or, in a heterologous manner, of the proteins of the invention with other proteins, for example, but not exclusively, docking proteins, enzymes, receptors, ion channels, uncoupling proteins, or translation factors. Agents could also act on the physical interaction of the proteins of this invention with other proteins, which are required for protein function, for example, but not exclusively, their downstream signaling.

Methods for determining protein-protein interaction are well known in the art. For example binding of a fluorescently labeled peptide derived from a protein of the invention to the interacting protein (or vice versa) could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known in the art, for example but not exclusively AlphaScreen (PerkinElmer) or Scintillation Proximity Assays (SPA) by Amersham. Alternatively, the interaction of the proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI. In all cases the two or more binding partners can be different proteins with one being the protein of the invention, or in case of dimerization or/and oligomerization the protein of the invention itself. Proteins of the invention, for which one target mechanism of interest, but not the only one, would be such protein/protein interactions are CG7042, astray, string, or CG1401 homologous proteins.

Assays for determining enzymatic activity of the proteins of the invention are well known in the art. Well known in the art are also a variety of assay formats to measure receptor-ligand binding or receptor downstream signalling.

For example, the method of radioligand binding for studying receptors is described by Keen M. (editor, (1998) Receptor Binding Techniques, Humana Press Inc.).

In addition, commercially available assays measure levels of cAMP. The assays are based on the competition between endogenous cAMP and exogenously added labeled cAMP. (e.g. AlphaScreen; PerkinElmer).

Alternatively, the calcium signalling could be the basis for a screening assay, in which calcium ion flux can be measured. For example, but not exclusively, widely applicated is a fluorescence-based assay system for the measurement of intracellular calcium developed by Molecular Devices. This application is, for example, described in Chambers C. et al., (2003) Comb Chem High Throughput Screen. 6: 355-362.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures or/and aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Candidate agents may also be found in phosphatase assays where a phosphatase substrate such as a protein, a peptide, a lipid, or an organic compound, which may or may not include modifications as further described below, or others are dephosphorylated by the proteins or protein fragments of the invention. A therapeutic candidate agent may be identified by its ability to increase or decrease the enzymatic activity of the proteins of the invention. The phosphatase activity may be detected by change of the chemical, physical or immunological properties of the substrate due to dephosphorylation. One example could be the cleavage of radioisotopically labelled phosphate groups from a phosphatase substrate catalyzed by the polypeptides of the invention. The dephosphorylation of the substrate may be followed by detection of the substrates autoradiography with techniques well known in the art.

Yet in another example, the change of mass of the substrate due to its dephosphorylation may be detected by mass spectrometry techniques. One could also detect the phosphorylation status of a substrate with an analyte discriminating between the phosphorylated and unphosphorylated status of the substrate. Such an analyte may act by having different affinities for the phosphorylated and unphosphorylated forms of the substrate or by having specific affinity for phosphate groups. Such an analyte could be, but is not limited to, an antibody or antibody derivative, a recombinant antibody-like structure, a protein, a nucleic acid, a molecule containing a complexed metal ion, an anion exchange chromatography matrix, an affinity chromatography matrix or any other molecule with phosphorylation dependend selectivity towards the substrate.

Such an analyte could be employed to detect the phosphatase substrate, which is immobilized on a solid support during or after an enzymatic reaction. If the analyte is an antibody, its binding to the substrate could be detected by a variety of techniques as they are described in Harlow and Lane, 1998, Antibodies, CSH Lab Press, NY. If the analyte molecule is not an antibody, it may be detected by virtue of its chemical, physical or immunological properties, being endogenously associated with it or engineered to it.

Yet in another example the phosphatase substrate may have features, designed or endogenous, to facilitate its binding or detection in order to generate a signal that is suitable for the analysis of the substrates phosphorylation status. These features may be, but are not limited to, a biotin molecule or derivative thereof, a glutathione-S-transferase moiety, a moiety of six or more consecutive histidine residues, an amino acid sequence or hapten to function as an epitope tag, a fluorochrome, an enzyme or enzyme fragment. The phosphatase substrate may be linked to these or other features with a molecular spacer arm to avoid steric hindrance.

In one example, the phosphatase substrate may be labelled with a fluorochrome. The binding of the analyte to the labelled substrate in solution may be followed by the technique of fluorescence polarization as it is described in the literature (see, for example, Parker, G. J. et al. (2000) J. Biomol. Screen. 5: 77-88). In a variation of this example, a fluorescent tracer molecule may compete with the substrate for the analyte to detect phosphatase activity by a technique which is known to those skilled in the art as indirect fluorescence polarization. A comercially available assay that utilizes an iron compound that acts as dark quencher upon specific binding to the phosphoryl group of a fluorescent dye-labeled phosphorylated peptide. The cleavage results in an increase in the observed fluorescence emission intensity of the dye-labeled peptide substrate after ii becomes dephosphorylated by the phosphatase (e.g. Pierce).

Another technique for drug screening, which may be used, provides for high throughput screening of compounds having suitable binding affinity to the protein of interest as described in published PCT application WO84/03564. In this method, as applied to the proteins of the invention large numbers of different small test compounds are synthesised on a solid substrate, such as plastic pins or some other surface. The test compounds are reacted with a protein of the invention, or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilise it on a solid support.

In another embodiment, one may use competitive drug screening assays in which neutralising antibodies capable of binding a protein of the invention specifically compete with a test compound for binding the protein. In this manner, the antibodies can be used to detect the presence of any peptide, which shares one or more antigenic determinants with the CG7042, astray, string, or CG1401 homologous protein.

The nucleic acids encoding the proteins of the invention can be used to generate transgenic animals or site-specific gene modifications in cell lines. These transgenic non-human animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test effectors/modulators of the proteins of the invention. Misexpression (for example, overexpression or lack of expression) of a protein of the invention, particular feeding conditions, or/and administration of biologically active compounds can create models of metablic disorders.

In one embodiment of the invention, such assays use mouse models of insulin resistance or/and diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al., 1998, supra). Susceptible wild type mice (for example C57BI/6) show similiar symptoms if fed a high fat diet. In addition to testing the expression of the proteins of the invention in such mouse strains (see Examples section), these mice could be used to test whether administration of a candidate effector/modulator alters for example lipid accumulation in the liver, in plasma, or adipose tissues using standard assays well known in the art, such as FPLC, colorimetric assays, blood glucose level tests, insulin tolerance tests and others.

Transgenic animals may be made through homologous recombination in non-human embryonic stem cells, where the normal locus of the gene encoding a protein of the invention is altered. Alternatively, a nucleic acid construct encoding a protein of the invention is injected into oocytes and is randomly integrated into the genome. Vectors for stable integration include plasmids, retroviruses and other animal viruses, yeast artificial chromosomes (YACs), and the like. The modified cells or animal are useful in the study of the function and regulation of the proteins of the invention. For example, a series of small deletions or/and substitutions may be made in the gene that encodes a protein of the invention to determine the role of particular domains of the protein, functions in pancreatic differentiation, etc.

Furthermore, variants of the genes of the invention like specific constructs of interest include anti-sense molecules, which will block the expression of the proteins of the invention, or expression of dominant negative mutations. A detectable marker, such as for example lac-Z or luciferase may be introduced in the locus of a gene of the invention, where up regulation of expression of the genes of the invention will result in an easily detected change in phenotype.

One may also provide for expression of the genes of the invention or variants thereof in cells or tissues where it is not normally expressed or at abnormal times of development. In addition, by providing expression of the proteins of the invention in cells in which they are not normally produced, one can induce changes in cell behavior,

DNA constructs for homologous recombination will comprise at least portions of the genes of the invention with the desired genetic modification, and will include regions of homology to the target locus. DNA constructs for random integration do not need to contain regions of homology to mediate recombination. Conveniently, markers for positive and negative selection are included. DNA constructs for random integration will consist of the nucleic acids encoding the proteins of the invention, a regulatory element (promoter), an intron and a poly-adenylation signal. Methods for generating cells having targeted gene modifications through homologous recombination are known in the art. For non-human embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF).

When non-human ES or embryonic cells or somatic pluripotent stem cells have been transfected, they may be used to produce transgenic animals. After transfection, the cells are plated onto a feeder layer in an appropriate medium. Cells containing the construct may be selected by employing a selective medium. After sufficient time for colonies to grow, they are picked and analyzed for the occurrence of homologous recombination or integration of the construct. Those colonies that are positive may then be used for embryo transfection and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a nucleic acid molecule coding for a protein of the invention or/and a functional fragment thereof;
(b) a protein of the invention or/and a functional fragment or/and an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or/and another effector/modulator of the nucleic acid of (a) or/and the polypeptide of (b), (e), or/and (f) and
(g) an anti-sense oligonucleotide of the nucleic acid of (a).

The kit may be used for diagnostic or therapeutic purposes or for screening applications as described above. The kit may further contain user instructions.

### THE FIGURES SHOW

FIG. 1 shows the triglyceride content of a Drosophila CG7042 (GadFly Accession Number) mutant. Shown is the change of triglyceride content of HD-EP(3)37139 flies caused by integration of the P-vector into the annotated transcription unit (referred to as ‘HD-EP37139’ in column 2) in comparison to controls containing all flies of the EP collection (referred to as ‘EP-control’, column 1).

FIG. 2 shows the molecular organization of the mutated CG7042 (Gadfly Accession Number) gene locus.

FIG. 3 shows the expression of the CG7042 (GadFly Accession Number) homolog in mammalian (mouse) tissues.

FIG. 3A shows the real-time PCR analysis of protein similar to DUAL-SPECIFICITY PHOSPHATASE TS-DSP1 (TS-DSP1) expression in wild-type mouse tissues.

FIG. 3B shows the real-time PCR analysis of TS-DSP1 expression in different mouse models.

FIG. 3C shows the real-time PCR analysis of TS-DSP1 expression in mice fed with a high fat diet compared to mice fed with a standard diet.

FIG. 3D shows the real-time PCR analysis of TS-DSP1 expression during the differentiation of 3T3-L1 cells from preadipocytes to mature adipocytes.

FIG. 4 shows the triglyceride content of Drosophila astray (GadFly Accession Number CG3705) mutants. Shown is the change of triglyceride content of HD-EP(3)36956 and HD-EP(3)36964 flies caused by integration of the P-vector into the annotated transcription unit (referred to as ‘HD-EP36956’ in column 2 and ‘HD-EP36964’ in column 3, respectively) in comparison to controls containing all flies of the EP collection (referred to as ‘EP-control’, column 1).

FIG. 5 shows the molecular organization of the mutated astray (aay; Gadfly Accession Number CG3705) gene locus.

FIG. 6 shows the expression of the astray homolog in mammalian (mouse) tissues.

FIG. 6A shows the real-time PCR analysis of phosophoserine phosphatase (Psph) expression in wild-type mouse tissues.

FIG. 6B shows the real-time PCR analysis of Psph expression in different mouse models.

FIG. 6C shows the real-time PCR analysis of Psph expression during the differentiation of 3T3-L1 cells from preadipocytes to mature adipocytes.

FIG. 7 shows the expression of the human astray homolog in mammalian (human) tissue. Shown is the microarray analysis of phosphoserine phosphatase (PSPH) expression in human adipocyte cells, during the differentiation from preadipocytes to mature adipocytes.

FIG. 8 shows the triglyceride content of a Drosophila string (GadFly Accession Number CG1395) mutant. Shown is the change of triglyceride content of HD-EP(3)36936 flies caused by integration of the P-vector into the annotated transcription unit (referred to as ‘HD-EP36936’ in column 2) in comparison to controls containing all flies of the EP collection (referred to as ‘EP-control’, column 1).

FIG. 9 shows the molecular organization of the mutated string (Gadfly Accession Number CG1 395) gene locus.

FIG. 10 shows the expression of a human string homolog in mammalian (human) tissue. Shown is the microarray analysis of cell division cycle 25B (CDC25B) expression in human adipocyte cells, during the differentiation from preadipocytes to mature adipocytes.

FIG. 11 shows the triglyceride content of a Drosophila CG1401 (GadFly Accession Number) mutant. Shown is the change of triglyceride content of HD-EP(3)36858 flies caused by integration of the P-vector into the annotated transcription unit (referred to as ‘HD-EP36858’ in column 2) in comparison to controls containing all flies of the. EP collection (referred to as ‘EP-control’, column 1).

FIG. 12 shows the molecular organization of the mutated CG1401 (Gadfly Accession Number) gene locus.

FIG. 13 shows the expression of the CG1401. GadFly Accession Number) homolog in mammalian (mouse) tissues.

FIG. 13A shows the real-time PCR analysis of RIKEN cDNA 4921514I20 gene (4921514I20Rik) expression in wild-type mouse tissues.

FIG. 13B shows the real-time PCR analysis of 4921514I20Rik expression in different mouse models.

FIG. 13C shows the real-time PCR analysis of 4921514I20Rik expression in mice fed with a high fat diet compared to mice fed with a standard diet.

FIG. 13D shows the real-time PCR analysis of 4921514I20Rik expression during the differentiation of 3T3-L1 cells from preadipocytes to mature adipocytes.

FIG. 14 shows the expression of the human CG1401 (GadFly Accession Number) homolog in mammalian (human) tissue. Shown is the microarray analysis of cullin 5 (CUL5) expression in human adipocyte cells, during the differentiation from preadipocytes to mature adipocytes.

### THE EXAMPLES ILLUSTRATE THE INVENTION

### EXAMPLE 1

### Measurement of Triglyceride Content

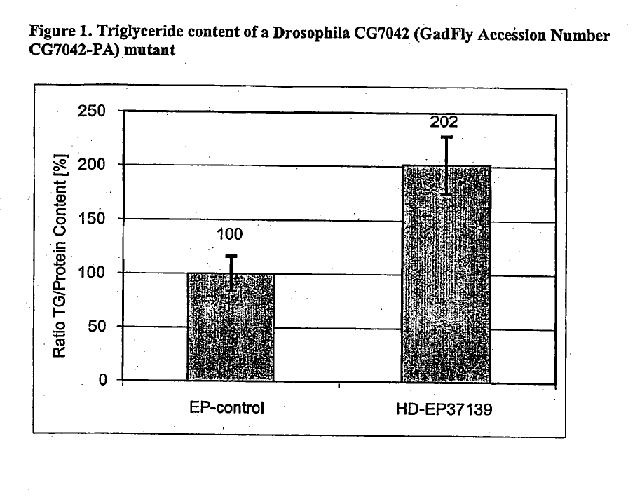

Mutant flies are obtained from a fly mutation stock collection. The flies are grown under standard conditions known to those skilled in the art. In the course of the experiment, additional feedings with bakers yeast (Saccharomyces cerevisiae) are provided for the EP-lines HD-EP(3)37139, HD-EP(3)36956, HD-EP(3)36964, HD-EP(3)36936, and HD-EP(3)36858. The average change of triglyceride content of Drosophila containing the EP-vector as homozygous viable integration was investigated in comparison to control flies (see FIGS. 1, 4, 8, and 11, respectively). For determination of triglyceride content, flies were, incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference the protein content of the same extract was measured using BIO-RAD DC Protein Assay according to the manufacturer's protocol. These experiments and assays were repeated several times.

The average triglyceride level of all flies of the EP collection (referred to as ‘EP-control’) is shown as 100% in the first columns in FIGS. 1, 4, 8, and 11. Standard deviations of the measurements are shown as thin bars.

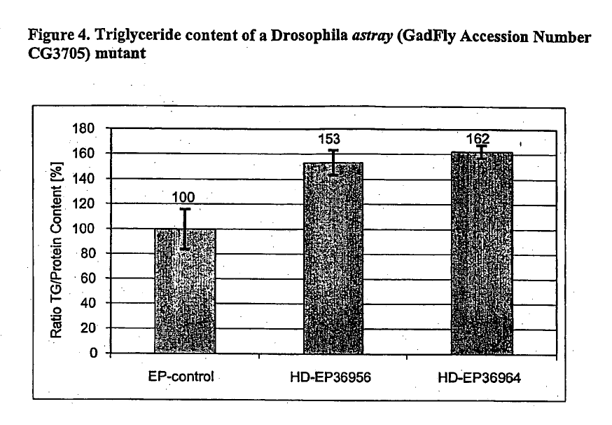

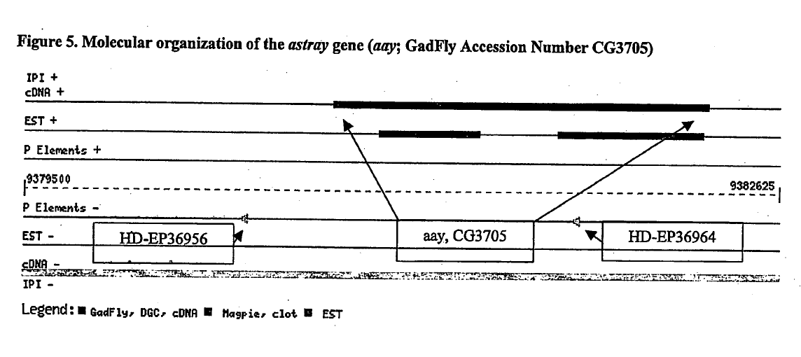

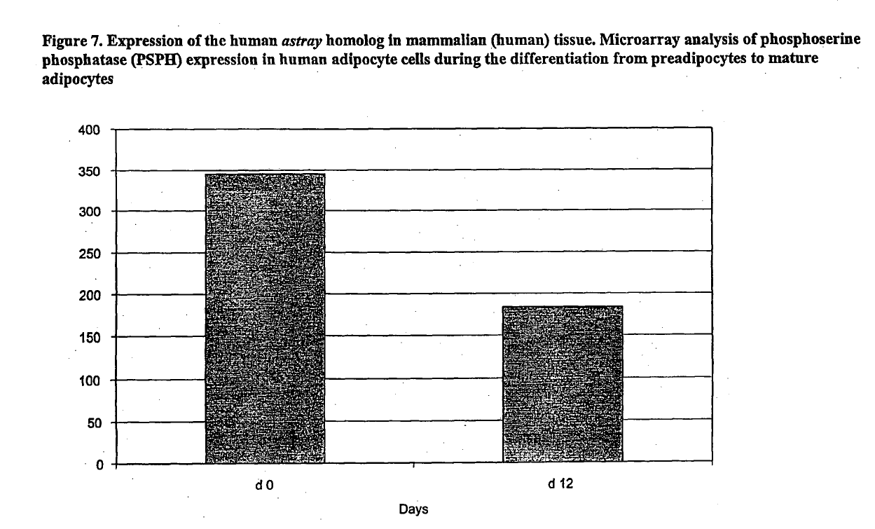

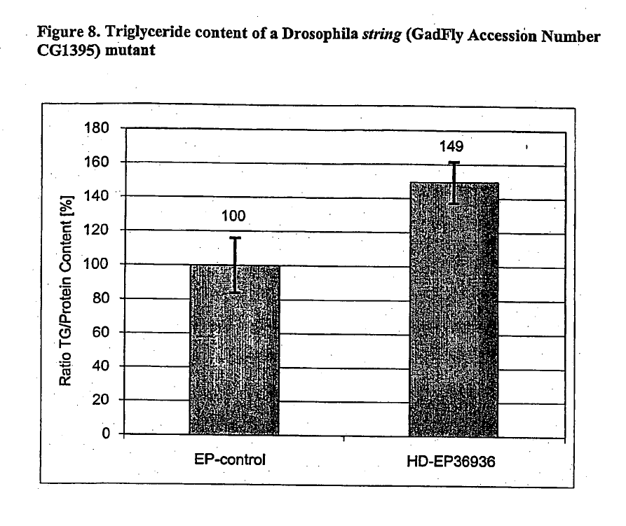

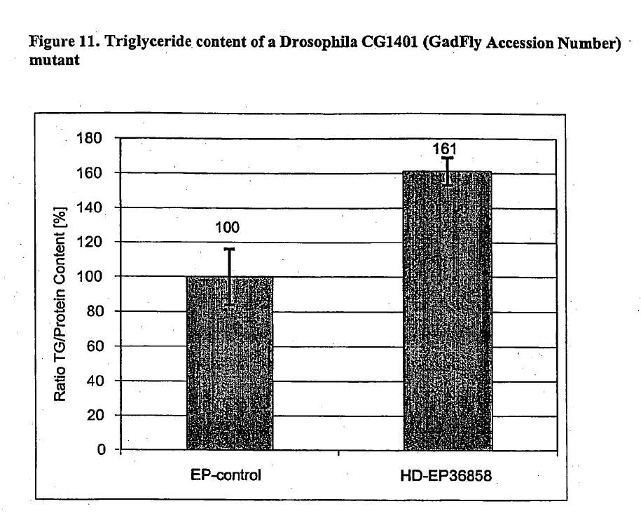

HD-EP(3)37139 homozygous flies (column 2 in FIG. 1, ‘HD-EP37139’), HD-EP(3)36956 and HD-EP(3)36964 homozygous flies (column 2 in FIG. 4, ‘HD-EP36956’, and column 3 in FIG. 4 ‘HD-EP36964’), HD-EP(3)36936 homozygous flies (column 2 in FIG. 8, ‘HD-EP36936’), and HD-EP(3)36858 homozygous flies (column 2 in FIG. 11, ‘HD-EP36858’) show constantly a higher triglyceride content than the controlsconstantly a higher triglyceride content than the controls (column 2 in FIG. 5, ‘HD-EP36936’). HD-EP(3) 36858 homozygous flies show constantly a higher triglyceride content than the controls (column 2 in FIG. 7, ‘HD-EP36858’). Therefore, the loss of gene activity is responsible for changes in the metabolism of the energy storage triglycerides.

### EXAMPLE 2

### Identification of Drosophila Genes Associated with Metabolic Regulation

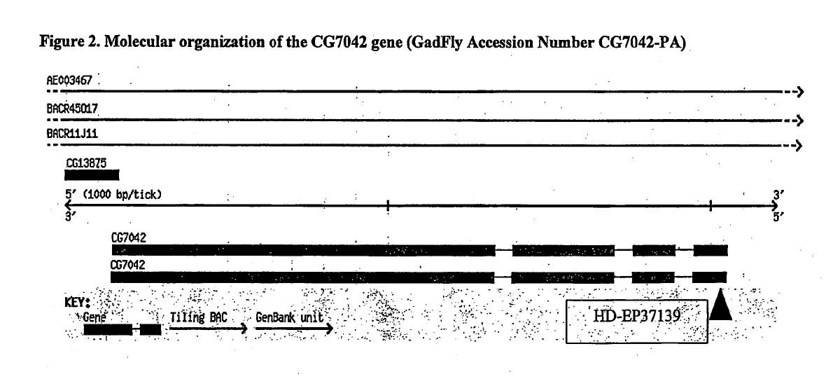

Nucleic acids encoding the proteins of the present invention were identified using a plasmid-rescue technique. Genomic DNA sequences were isolated that are localized adjacent to the EP vector (herein HD-EP(3)37139, HD-EP(3) 36956, HD-EP(3)36964, HD-EP(3)36936, and HD-EP(3)36858) integration. Using those isolated genomic sequences public databases like Berkeley Drosophila Genome Project (GadFly) were screened, thereby identifying the integration sites of the vectors, and the corresponding genes. The molecular organization of these gene loci is shown in FIGS. 2, 5, 9, and 12.

The HD-EP(3)37139 vector is homozygous viable integrated into the leader sequence of cDNA CG7042-RA and into the cDNA CG7042-RB at base pair 49 in sense orientation. The chromosomal localization site of integration of the vector of HD-EP(3)37139 is at gene locus 3L, 61B2. in FIG. 2, genomic DNA sequence is represented by the assembly as a black scaled double-headed arrow in middle of the figure that includes the integration site of HD-EP(3) 37139. Ticks represent the length in basepairs of the genomic DNA (1000 base pairs per tick). The grey arrows in the upper part of the figure represent BAC clones, the black arrow in the topmost part of the figure represents the section of the chromosome. The insertion site of the P-element in the Drosophila line HD-EP37139 is shown as a black triangle in the lower half of the figure and is labeled. The cDNA sequences of the predicted genes (as predicted by the Perkeley Drosophila Genome Project, GadFly release 3) are shown as dark grey bars (exons), linked by dark grey lines (introns), and are labeled (see also key at the bottom of the figure). The predicted cDNAs of the Drosophila CG7042 gene (GadFly Accession Number) are shown in the lower half of the figure.

In FIGS. 5, 9, and 12, genomic DNA sequence is represented by the assembly as a dotted black line in the middle that includes the integration sites of the vectors for lines HD-EP(3)36956, HD-EP(3)36964, HD-EP(3)36936, or HD-EP(3)36858. Numbers represent the coordinates of the genomic DNA. The upper parts of the figures represent the sense strand “+” the lower parts represent the antisense strand “−”. The insertion sites of the P-elements in the Drosophila lines are shown as triangles or boxes in the “P-elements +” or/and “P-elements −” lines. Transcribed DNA sequences (ESTs) are shown as grey bars in the “EST +”, “EST −”, “IPI +”, or/and the “IPI −” lines, and predicted cDNAs are shown as bars in the “cDNA+” and/or “cDNA −” lines. Predicted exons of the cDNAs are shown as dark grey bars and predicted introns are shown as light grey bars (see also legend at the bottom of the figures).

The HD-EP(3)36956 vector is homozygous viable integrated 370 base pairs 5′ of CG3705-RA in antisense orientation, and the HD-EP(3)36964 vector is homozygous viable integrated 1003 base pairs 3′ of the transcription start of CG3705-RA in antisense orientation, identified as astray (referred to as aay; GadFly Accession Number CG3705). The chromosomal localization site of integration of the vectors HD-EP(3)36956 and HD-EP(3)36964 is at gene locus 3L, 67B1 (according to FlyBase), 67B4 (according to GadFly release 3). In FIG. 5, the coordinates of the genomic DNA start at position 9379500 on chromosome 3L, ending at position 9382625. The insertion sites of the P-elements in Drosophila HD-EP(3)36956 and HD-EP(3)36964 lines are shown as triangles in the “P Elements −” line and are labeled. The predicted cDNA of the astray gene shown in the “cDNA +” line is labeled (referred to as aay, CG3705). The corresponding ESTs are shown in the “EST +” line.

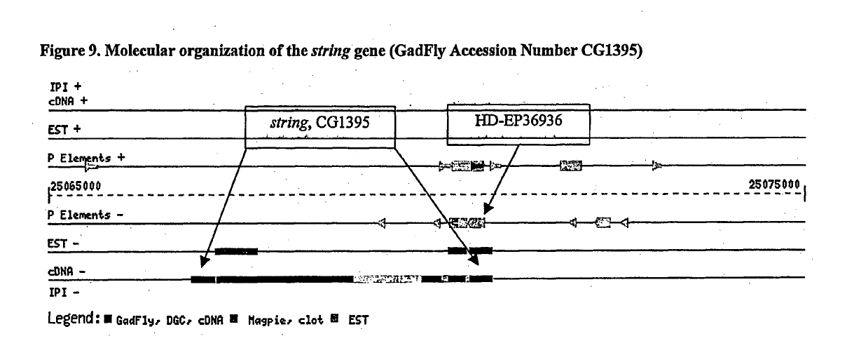

The HD-EP(3)36936 vector is homozygous viable integrated into the cDNA at base pair 144 of a Drosophila gene in sense orientation identified as string (GadFly Accession Number CG1395). The chromosomal localization site of integration of the vector HD-EP(3)36936 is at gene locus 3R, 98F1 3 (according to FlyBase), 99A5 (according to GadFly release 3). In FIG. 9, the coordinates of the genomic DNA start at position 25065000 on chromosome 3R, ending at position 25075000. The insertion site of the P-element in Drosophila HD-EP(3) 36936 line is shown as triangle in the “P Elements −” line and is labeled. The predicted cDNA of the string gene shown in the “cDNA −” line is labeled (referred to as sting, CG1395). The corresponding ESTs are shown in the “EST −” line.

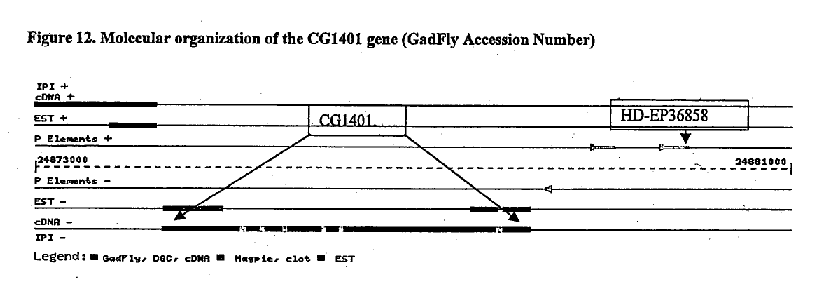

The HD-EP(3)36858 vector is homozygous viable integrated 1663 base pairs 5′ of the cDNA of a Drosophila gene in antisense orientation, identified as CG1401-RA (referred to as GadFly Accession Number CG1401). The chromosomal localization site of integration of the vector HD-EP(3)36858 is at gene locus 3R, 98F4 (according to FlyBase), 98F6 (according to GadFly release 3). In FIG. 12, the coordinates of the genomic DNA start at position 24873000 on chromosome 3R, ending at position 24873000. The insertion site of the P-element in Drosophila HD-EP(3)36858 line is shown as box in the “P Elements +” line and is labeled. The predicted cDNA of the CG1401 gene shown in the “cDNA −” line is labeled. The corresponding ESTs are shown in the “EST −” line.

Expression of the genes described above could be effected by integration of the vectors into the transcription units, leading to a change in the amount of the energy storage triglycerides.

### EXAMPLE 3

### Identification of Human Homologous Genes and Proteins

The Drosophila genes and proteins encoded thereby with functions in the regulation of triglyceride metabolism were further analysed using the BLAST algorithm searching in publicly available sequence databases and mammalian homologs were identified (see Table 1).

The term “polynucleotide comprising the nucleotide sequence as shown in GenBank Accession number” relates to the expressible gene of the nucleotide sequences deposited under the corresponding GenBank Accession number. The term “GenBank Accession number” relates to NCBI GenBank database entries (Ref.: Benson et al., (2000) Nucleic Acids Res. 28: 15-18). Sequences homologous to Drosphila CG7042, astray, string, and CG1401 were identified using the publicly available program BLASTP 2.2.3 of the non-redundant protein data base of the National Center for Biotechnology Information (NCBI) (see, Altschul S. F. et al., (1997) Nucleic Adds Res. 25: 3389-3402).

TABLE 1
Human homologs of the Drosophila (Dm) genes

| Dm gene | Homo sapiens homologous genes and proteins | | |
|---|---|---|---|
| Acc. No. | Accession Number | | |
| Name | cDNA | Protein | Name |
| CG7042 | NM_080876 | NP_543152 | dual specificity phosphatase 19 |
| | | | (DUSP19); stress-activated protein |
| | | | kinase pathway-regulating phosphatase 1 |
| CG3705 | NM_004577 | NP_004568 | phosphoserine phosphatase (PSPH); |
| astray | | | PSPase |
| CG1395 | NM_001789 | | cell division cycle 25A |
| string | | | (CDC25A) |
| | NM_004358 | NP_004349 | cell division cycle 25B (CDC25B) |
| | | | isoform 1 |
| | NM_021872 | NP_068658 | cell division cycle 25B (CDC25B) |
| | | | isoform 2 |
| | NM_021873 | NP_068659 | cell division cycle 25B (CDC25B) |
| | | | isoform 3 |
| | NM_021874 | NP_068660 | cell division cycle 25B (CDC25B) |
| | | | isoform 4 |
| | NM_001790 | NP_001781 | cell division cycle 25C (CDC25C) |
| | | | protein isoform a |
| | | | protein isoform b |
| | NM_003478 | NP_003469 | |

CG7042, astray, string, or CG1401 homologous proteins and nucleic acid molecules coding therefore are obtainable from insect or vertebrate species, e.g. mammals or birds. Particularly preferred are nucleic acids as described in Table 1).

Human dual specificity phosphatase 19 is also referred to in patent applications WO01/73060, WO01/12819, WO01/81590, and WO00/60099. Human cell division cycle 25A is also referred to in patent applications WO93/10242, WO02/070680, and WO01/27077. Human cell division cycle 25 C is also referred to in patent applications WO96/12820, EP1096014, WO01/16300, and WO98/30680.

The mouse homologous cDNAs encoding the polypeptides of the invention were, identified as GenBank Accession Numbers NM_{—}024438 (for the mouse homolog to CG7042; Mm dual specificity phosphatase 1.9), NM_{—}133900 (for the mouse homolog to astray; Mm expressed sequence A1480570), NM_{—}007658 (for the mouse homolog to string; Mm cell division cycle 25 homolog A, Cdc25a), NM_{—}023117 (for the mouse homolog to string; Mm cell division cycle 25 homolog B, Cdc25b), NM_{—}009860 (for the mouse homolog to string; Mm cell division cycle 25 homolog C, Cdc25c), XM_{—}134805 (for the mouse homolog to CG1401; Mm RIKEN cDNA 4921514120 gene).

### EXAMPLE 4

### Expression of the Polypeptides in Mammalian (Mouse) Tissues

To analyse the expression of the polypeptides disclosed in this invention in mammalian tissues, several mouse strains, (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C.), 40 per cent humidity and a light/dark cycle of preferrably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum (see, for example, Schnetzler B. et al., 1993, J Clin Invest 92: 272-280, Mizuno T. M. et al., 1996, Proc Natl Acad Sci U S A 93: 3434-3438). In a further experiment wild-type (wt) mice were fed a control diet (preferably Altromin C1057 mod control, 4.5% crude fat) or high fat diet (preferably Altromin C1057 mod. high fat, 23.5% crude fat). Animals were sacrificed at an age of 6 to 8 weeks. The animal tissues were isolated according to, standard procedures known to those skilled in the art, snap frozen in liquid nitrogen and stored at −80° C. until needed.

For analyzing the role of the proteins disclosed in this invention in the in vitro differentiation of mammalian cell culture cells for the conversion of pre-adipocytes to adipocytes, mammalian fibroblast (3T3-L1) cells (e.g., Green H. and Kehinde O., 1974, Cell 1: 113-116) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu Z. et al., 2001, J. Biol. Chem. 276: 11988-11995; Slieker L. J. et al., 1998, BBRC 251: 225-229). In brief, cells were plated in DMEM/10% FCS (invitrogen, Karlsruhe, Germany) at 50,000 cells/well in duplicates in 6-well plastic dishes and cultured in a humidified atmosphere of 5% CO₂ at 37° C. At confluence (defined as day 0: d0) cells were transferred to serum-free (SF) medium, containing DMEM/HamF12 (3:1; Invitrogen), Fetuin (300microg/ml; Sigma, Munich, Germany), Transferrin (2 microg/ml; Sigma), Pantothenate (17 microM; Sigma), Biotin (1 microM; Sigma), and EGF (0.8 nM; Hoffmann-La Roche, Basel, Switzerland). Differentiation was induced by adding Dexamethasone (DEX; 1 microM; Sigma), 3-Methyl-Isobutyl-1-Methylxanthine (MIX; 0.5 mM; Sigma), and bovine Insulin (5 microg/ml; Invitrogen). Four days after confluence (d4), cells were kept in SF medium, containing bovine insulin (5 microg/ml) until differentiation was completed. At various time points of the differentiation procedure, beginning with day 0 (day of confluence) and day 2 (hormone addition; for example, dexamethason and 3-isobutyl-1-methylxanthin), up to 10 days of differentiation, suitable aliquots of cells were taken every two days.

RNA was isolated from tissues and cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to la those skilled in the art. Total RNA was reverse transcribed (preferrably using Superscript II RNaseH⁻ Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

Taqman analysis was performed preferably using the following primer/probe pairs:

For the amplification of mouse protein similar to dual-specificity phosphatase TS-DSP1 (TS-DSP1) sequence (GenBank Accession Number AK018369): Mouse TS-DSP1 forward primer (SEQ ID NO: 1): 5′-ACT GCC CTG TCG TTG GTG A-3′; mouse TS-DSP1 reverse primer (SEQ ID NO: 2): 5′-AGT TGT TCC ATG MG CCA GGA-3′; mouse TS-DSP1 Taqman probe (SEQ ID NO: 3): (5/6-FAM)-AGA GGC GAG ACC ATC CAT ATG TCC GA (5/6-TAMRA).

For the amplification of mouse phosphoserine phosphatase (Psph) sequence (GenBank Accession Number NM_{—}133900):

Mouse Psph forward primer (SEQ ID NO: 4): 5′-TGG CAC TGA TCC AGC CCT-3′; mouse Psph reverse primer (SEQ ID NO: 5): 5′-TCA GAT GTG GCG GGT GCT-3′; mouse Psph Taqman probe (SEQ ID NO: 6): (5/6-FAM)-CAG GGA TCA AGT CCA GAG GCT CCT AGC T-(516-TAMRA).

For the amplification of mouse RIKEN cDNA 4921514120 gene (4921514I20Rik) sequence (GenBank Accession Number XM_{—}134805): Mouse 4921514I20Rik forward primer (SEQ ID NO: 7): 5′-TTG CM CGG AAC TCC CAG A-3′; mouse 4921514I20Rik reverse primer (SEQ ID NO: 8): 5′-TGG GTG AGT TGA CTT GAG GGT C-3′; mouse 4921514I20Rik Taqman probe (SEQ ID NO: 9): (5/6-FAM)-TAG TAG CTT TTC CCA AGC TCA AAC GGC MG-(5/6-TAMRA).

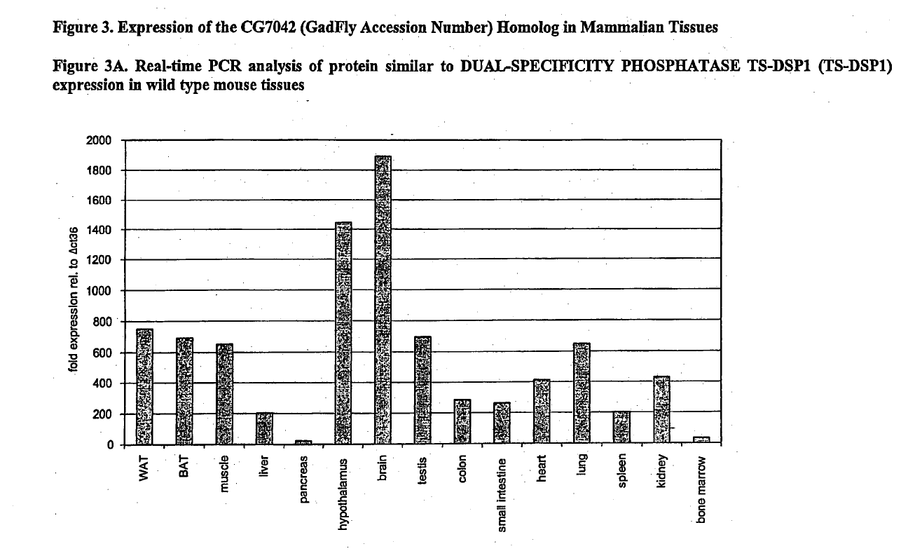

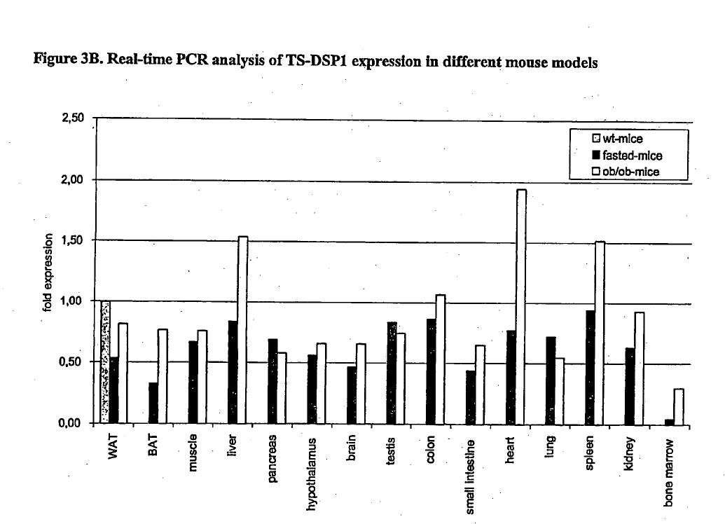

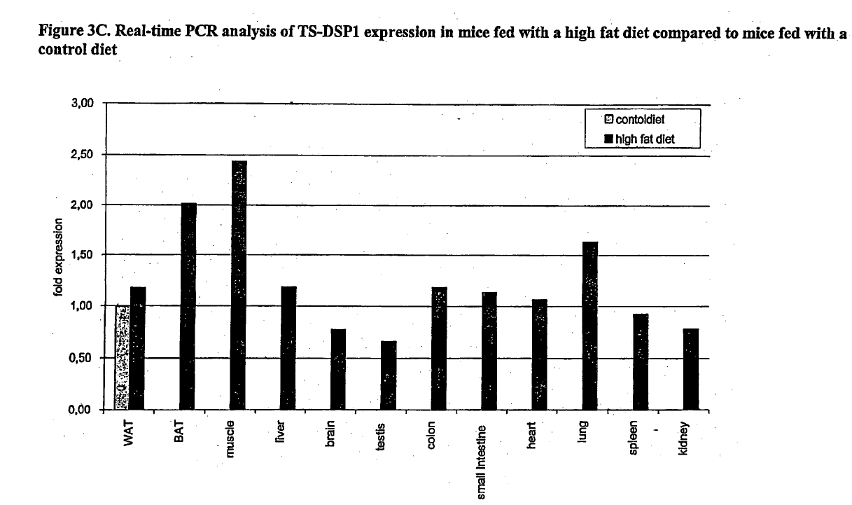

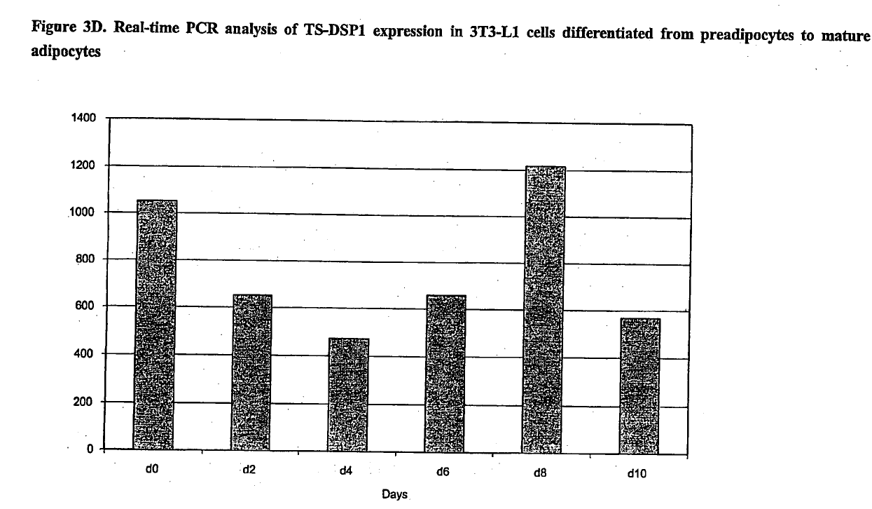

In the figures the relative RNA-expression is shown on the Y-axis. In FIGS. 3A-C, 6A-B, and 13A-C, the tissues tested are given on the X-axis. “WAT” refers to white adipose tissue, “BAT” refers to brown adipose tissue. In FIG. 3D, 6C, and 13D, the X-axis represents the time axis. “d0” refers to day 0 (start of the experiment), “d2”-“d10” refers to day 2-day 10 of adipocyte differentiation.

The function of the proteins of the invention in metabolism was further validated by analyzing the expression of the transcripts in different tissues and by analyzing the role in adipocyte differentiation.

In one embodiment of this invention, mouse models of insulin resistance or/and diabetes were used, such as mice carrying gene knockouts in the leptin pathway (for example, ob/ob (leptin) or db/db (leptin receptor/ligand) mice) to study the expression of the proteins of the invention. Such mice develop typical symptoms of diabetes, show hepatic lipid accumulation and frequently have increased plasma lipid levels (see Bruning J. C. et al., (1998) Mol. Cell. 2: 559-569).

In a further embodiment of the invention, expression of the mRNAs encoding the proteins of the invention was also examined in susceptible wild type mice (for example, C57BI/6) that show symptoms of diabetes, lipid accumulation, and high plasma lipid levels, if fed a high fat diet.

Expression profiling studies confirm the particular relevance of the proteins of the present invention as regulators of energy metabolism in mammals.

Taqman analysis revealed that the protein similar to dual-specificity phosphatase TS-DSP1 (TS-DSP1) is expressed in several mammalian tissues, showing highest level of expression in brain and hypothalamus and higher levels in further tissues, e.g. white adipose tissue (WAT), brown adipose tissue (BAT), muscle, testis, and lung. Furthermore TS-DSP1 is expressed on lower but still robust levels in liver, colon, small intestine, heart, spleen, and kidney. A significant expression is also detectable in pancreas and bone marrow of wild type mice as depicted in FIG. 3A. We found, for example, that the expression of TS-DSP1 is down regulated in the BAT, brain, small intestine and bone marrow of fasted mice compared to wild type mice. Furthermore the expression of TS-DSP1 is down regulated in the bone marrow of genetically induced obese mice (ob/ob) compared to wild type mice. (see FIG. 3B). In wild type mice fed a high fat diet, the expression of TS-DSP1 is up regulated in BAT and muscle, as depicted in FIG. 3C. We show in this invention (see FIG. 3D) that the TS-DSP1 mRNA is expressed and regulated during the differentiation into mature adipocyctes. Therefore, the TS-DSP1 protein might play a role in adipogenesis.

The expression of TS-DSP1 in metabolic active tissues of wild type mice, as well as the regulation of TS-DSP1 in different animal models used to study metabolic disorders, suggests that this gene plays a central role in energy homeostasis. This hypothesis is supported by the expression during the differentiation from preadipocytes to mature adipocytes.

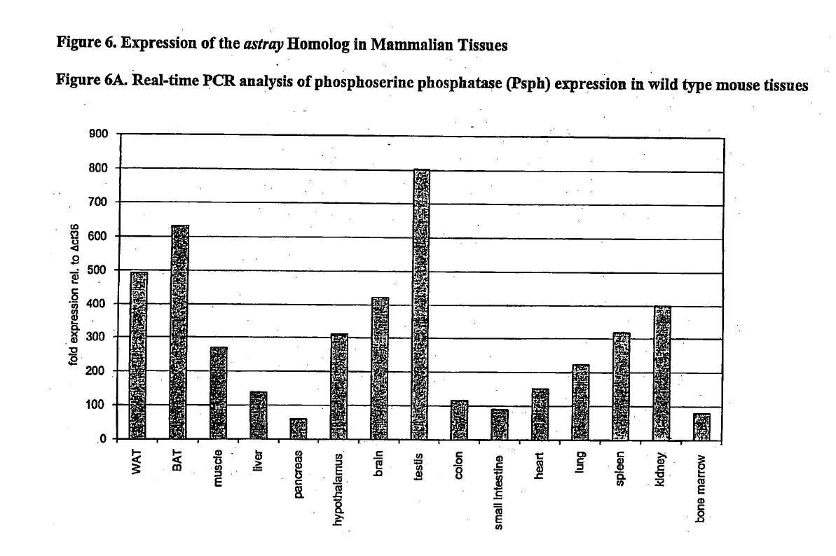

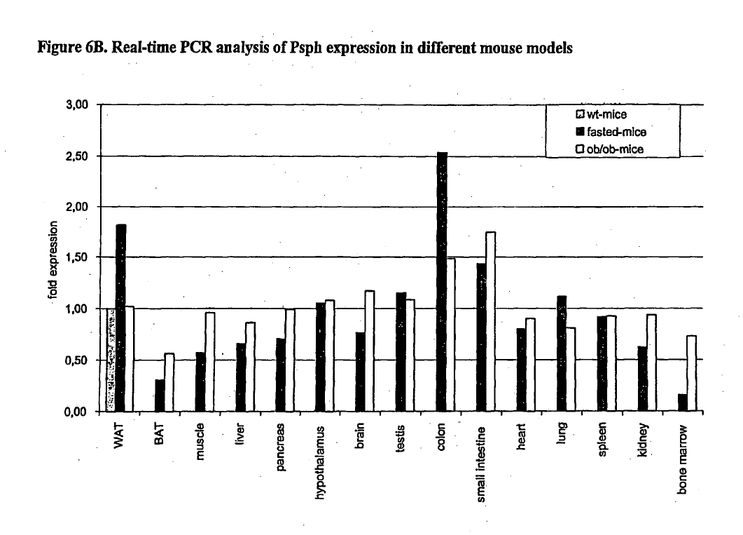

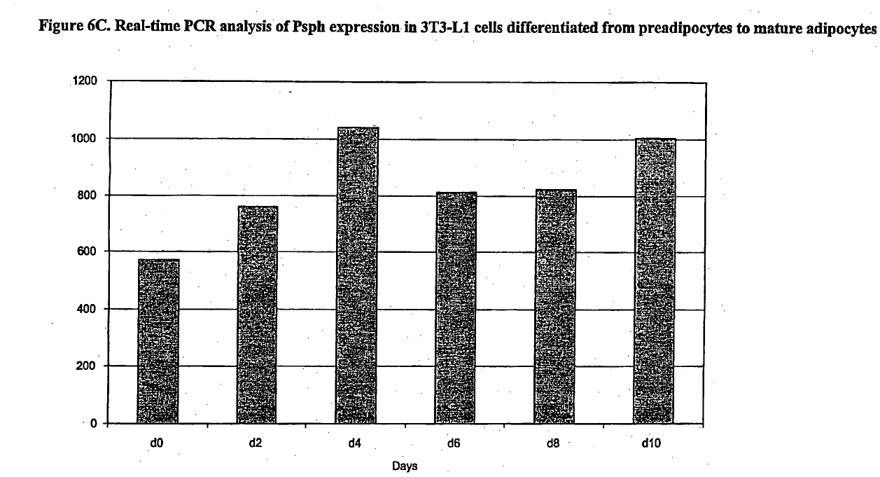

Taqman analysis revealed that phosphoserine phosphatase (Psph) is expressed in several mammalian tissues, showing highest level of expression in testis, WAT and BAT and higher levels in further tissues, e.g. muscle, liver, hypothalamus, brain colon, heart, lung, spleen and kidney of wild type mice. Furthermore Psph is expressed on lower but still robust levels in small intestine, pancreas and bone marrow of wild type mice as depicted in FIG. 6A. We found, for example, that the expression of Psph is down regulated in the BAT and bone marrow and up regulated in the colon of fasted mice compared to wild type mice (see FIG. 6B). We show in this invention (see FIG. 6C) that the Psph mRNA is expressed and up regulated during the differentiation into mature adipocyctes. Therefore, the Psph protein might play a role in adipogenesis.

The regulated expression of Psph in an animal model used to study metabolic disorders, together with the up regulation during the differentiation from preadipocytes to mature adipocytes, suggests that this gene plays a central role in energy homeostasis.

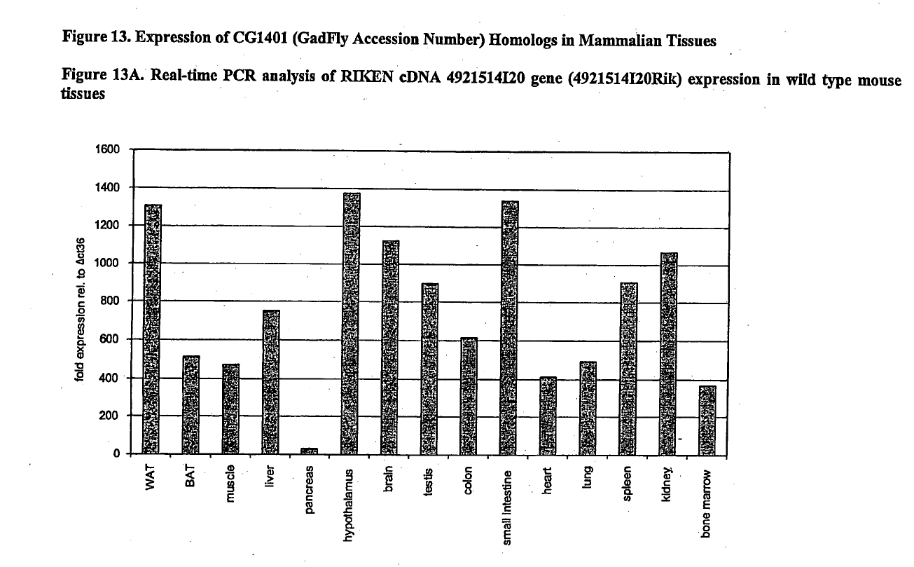

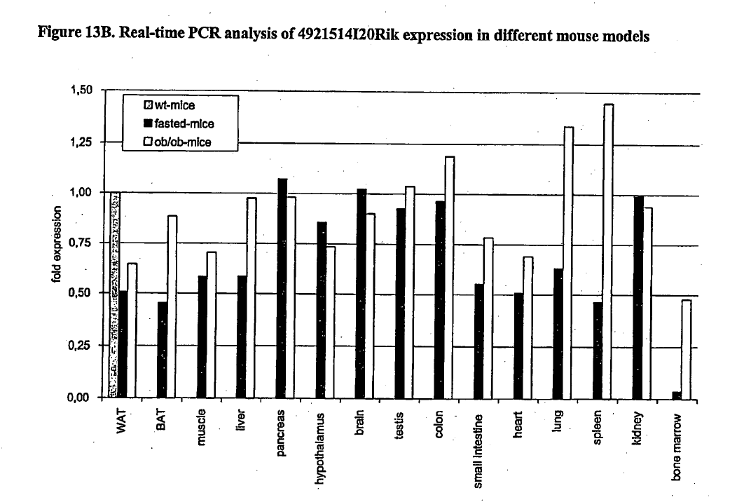

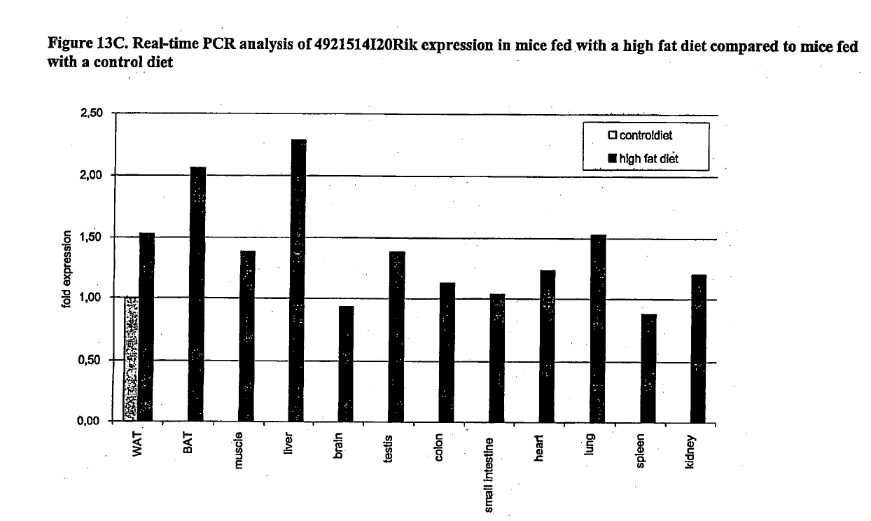

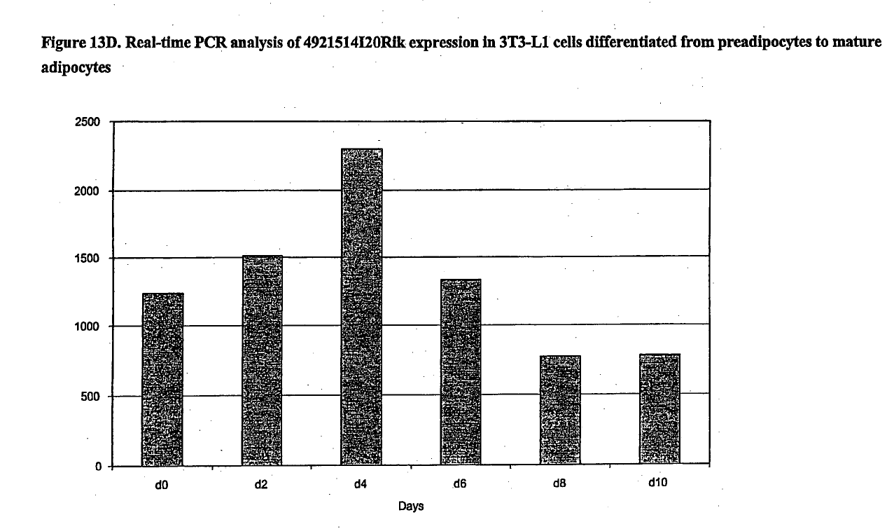

Taqman analysis revealed that RIKEN cDNA 4921514I20 gene (4921514I20Rik) is expressed in several mammalian tissues, showing highest level of expression in WAT, hypothalamus, and small intestine and higher levels in further tissues, e.g. liver, brain, testis, colon, spleen, and kidney. Furthermore 4921514I20Rik is expressed on lower but still robust levels in BAT, muscle, heart, lung, and bone marrow of wild type mice as depicted in FIG. 13A. We found, for example, that the expression of 4921514I20Rik is down regulated in the bone marrow of genetically induced obese mice (ob/ob) compared to wild type mice. Furthermore 4921.514I20Rik is down regulated in BAT, spleen and bone marrow of fasted mice compared to wild type mice (see FIG. 13B). In wild type mice fed a high fat diet, the expression of 4921514I20Rik is up regulated in BAT and in liver as depicted in FIG. 13C. We show in this invention (see FIG. 13D) that the 4921514I20Rik mRNA is expressed and transiently up regulated during the differentiation into mature adipocyctes. Therefore, the 4921514I20Rik protein might play an essential role in adipogenesis.

The expression of 4921514I20Rik is regulated in metabolic active tissues (e.g. BAT and liver) of different animal models used to study metabolic disorders, together with the regulated expression during the differentiation from preadipocytes to mature adipocytes, suggests that this gene plays a central role in energy homeostasis.

### EXAMPLE 5

### Analysis of the Differential Expression of Transcripts of the Proteins of the Invention in Human Tissues

RNA preparation from human primary adipose tissues was done as described in Example 4. The target preparation, hybridization, and scanning was performed as described in the manufactures manual (see Affymetrix Technical Manual, 2002, obtained from Affmetrix, Santa Clara, USA).

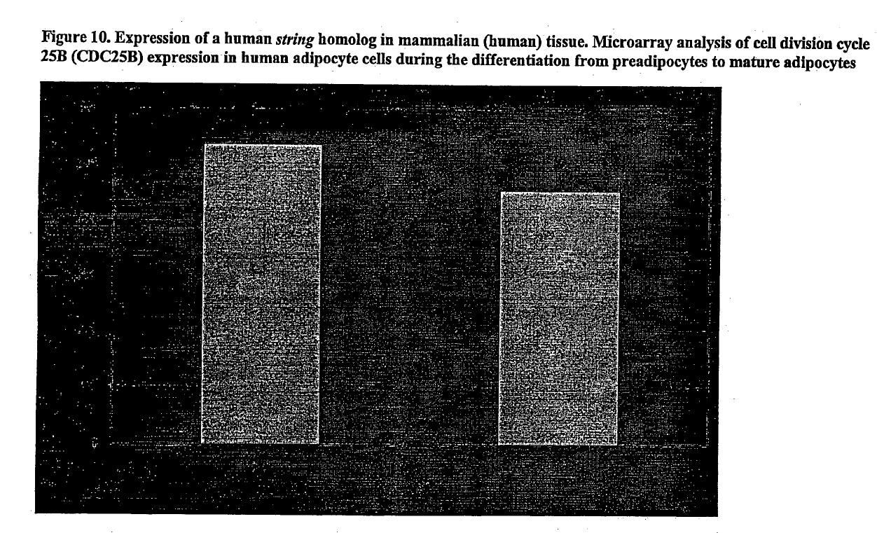

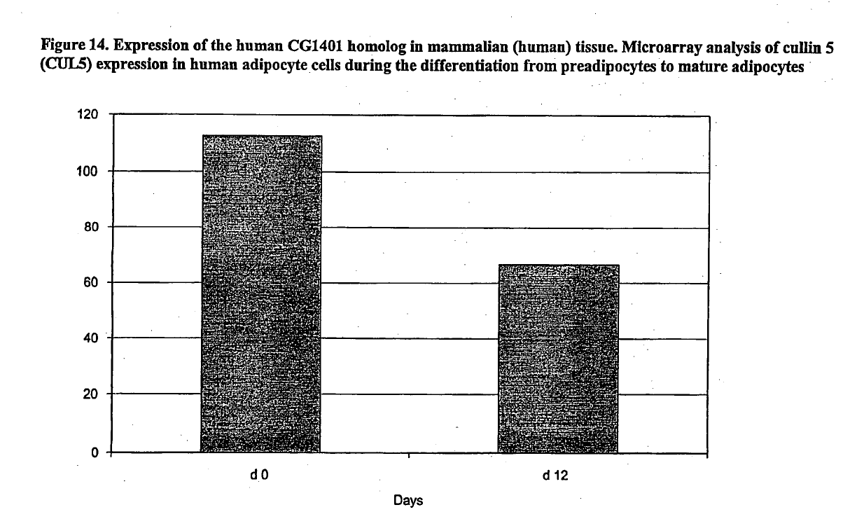

In FIGS. 7, 10, and 14, the X-axis represents the time axis, shown are day 0 and day 12 of adipocyte differentiation. The Y-axis represents the flourescent intensity. The expression analysis (using Affymetrix GeneChips) of the phosphoserine phosphatase (PSPH), cell division cycle 25B (CDC25B), and cullin 5 (CUL5) genes using human adipocyte cell line (SGBS) differentiation, clearly shows differential expression of human PSPH, CDC25B, and CUL5 genes in adipocytes. Several independent experiments were done. The experiments further show that the PSPH, CDC25B, and CUL5 transcript (see FIGS. 7, 10, and 14) is most abundant at day 0 compared to day 12 during differentiation.

Thus, the PSPH, CDC25B, and CUL5 proteins have to be significantly decreased in order for the preadipocyctes to differentiate into mature adipocycte. Therefore, PSPH, CDC25B, and CUL5 in preadipocyctes have the potential to inhibit adipose differentiation. Therefore, PSPH, CDC25B, and CUL5 proteins might play an essential role in the regulation of human metabolism, in particular in the regulation of adipogenesis and thus it might play an essential role in obesity, diabetes, or/and metabolic syndrome.

For the purpose of the present invention, it will be understood by the person having average skill in the art that any combination of any feature mentioned throughout the specification is explicity disclosed herewith.

1. A pharmaceutical composition comprising a CG7042, astray, string, or/and CG61401 homologous protein or/and a functional fragment thereof, a nucleic acid molecule encoding a CG7042, astray, string, or CG61401 homologous protein or/and a functional fragment thereof or/and a modulator/effector of said nucleic acid molecule or/and said protein together with pharmaceutically acceptable carriers, diluents or/and additives.

2. The composition of claim 1, wherein the nucleic acid molecule is a vertebrate or insect CG7042, astray, string, or CG1401 nucleic acid, particularly encoding a human protein as described in Table 1, or/and a nucleic acid molecule which is complementary thereto or a functional fragment thereof or a variant thereof.

3. The composition of claim 1 or 2, wherein said nucleic acid molecule is selected from the group consisting of
(a) a nucleic acid molecule encoding a polypeptide as shown in Table 1, or/and an isoform, fragment, or/and variant of said polypeptide;
(b) a nucleic acid molecule which comprises or is the nucleic acid molecule as shown in Table 1;
(c) a nucleic acid molecule being degenerated as a result of the genetic code to the nucleic sequence as defined in (a) or (b);
(d) a nucleic acid molecule that hybridizes at 50° C. in a solution containing 1×SSC and 0.1% SDS to a nucleic acid molecule as defined in claim 2 or as defined in (a) to (c) or/and a nucleic acid molecule which is complementary thereto;
(e) a nucleic acid molecule that encodes a polypeptide which is at least 85%, preferably at least 90%, more preferably at least 95%, more preferably at least 98% and up to 99,6% identical to the human protein CG7042, astray, string or CG1401, preferably as described in Table 1 or as defined in claim 2 or to a polypeptide as defined in (a);
(f) a nucleic acid molecule that differs from the nucleic acid molecule of (a) to (e) by mutation and wherein said mutation causes an alteration, deletion, duplication or premature stop in the encoded polypeptide.

4. The composition of claim 3, wherein said nucleic acid molecule is a DNA molecule, particularly a cDNA or a genomic DNA.

5. The composition of claim 3, wherein said nucleic acid encodes a polypeptide contributing to regulating the energy homeostasis or/and the metabolism of triglycerides.

6. The composition of claim 3, wherein said nucleic acid molecule is a recombinant nucleic acid molecule.

7. The composition of claim 3, wherein the nucleic acid molecule is a vector, particularly an expression vector.

8. The composition of claim 3, wherein the polypeptide is a recombinant polypeptide.

9. The composition of claim 8, wherein said recombinant polypeptide is a fusion polypeptide.

10. The composition of claim 3, wherein said nucleic acid molecule is selected from hybridization probes, primers and anti-sense oligonucleotides.

11. The composition of claim 3, which is a diagnostic composition.

12. The composition of claim 3, which is a therapeutic composition.

13. The composition of claim 3, for the manufacture of an agent for detecting or/and verifying, for the treatment, alleviation or/and prevention of metabolic diseases or dysfunctions, including metabolic syndrome, obesity, or/and diabetes, as well as related disorders such as eating disorder, cachexia, hypertension, coronary heart disease, hypercholesterolemia, dyslipidemia, osteoarthritis, gallstones, or liver fibrosis, in cells, cell masses, organs or/and subjects.

14. The composition of claim 3, for application in vivo.

15. The composition of claim 3, for application in vitro.

16. Use of a nucleic acid molecule encoding a CG7042, astray, string, or CG1401 homologous protein or an isoform, a functional fragment or variant thereof, in particular a nucleic acid molecule as described in Table 1, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or polypeptide for the manufacture of a medicament for the treatment of obesity, diabetes, or/and metabolic syndrome for controlling the function of a gene or/and a gene product which is influenced or/and modified by a CG7042, astray, string, or CG1401 homologous polypeptide, particularly by a polypeptide according to claim 3.

17. Use of the nucleic acid molecule encoding a CG7042, astray, string, or CG1401 homologous protein or an isoform, a functional fragment or variant thereof, in particular a nucleic acid molecule as described in Table 1, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or said polypeptide for identifying substances capable of interacting with a CG7042, astray, string, or CG1401 homologous polypeptide, particularly with a polypeptide according to claim 3.

18. A non-human transgenic animal exhibiting a modified expression of a CG7042, astray, string, or CG1401 homologous polypeptide, particularly of a polypeptide according to claim 3.

19. A non-human transgenic animal exhibiting a modified expression of a CG7042, astray string, or CG1401 homologous polypeptide, particularly of a polypeptide according to claim 3, wherein the expression of the CG7042, astray, string, or CG1401 homologous polypeptide, particularly of a polypeptide according to claim 3, is increased or/and reduced.

20. A recombinant host cell exhibiting a modified expression of a CG7042, astray, string, or CG1401 homologous polypeptide, particularly of a polypeptide according to claim 3.

21. The cell of claim 20 which is a human cell.

22. A method of identifying a (poly)peptide involved in the regulation of energy homeostasis or/and metabolism of triglycerides in a mammal comprising the steps of
(a) contacting a collection of (poly)peptides with a CG7042, astray, string, or CG1401 homologous polypeptide, particularly a polypeptide according to claim 3, or a functional fragment thereof under conditions that allow binding of said (poly)peptides;
(b) removing (poly)peptides which do not bind and
(c) identifying (poly)peptides that bind to said CG7042, astray, string, or CG1401 homologous polypeptide.

23. A method of screening for an agent which modulates/effects the interaction of a CG7042, astray, string, or CG1401 homologous polypeptide, particularly of a polypeptide according to claim 3, with a binding target, comprising the steps of
(a) incubating a mixture comprising
(aa) a CG7042, astray, string, or CG1401 homologous polypeptide, particularly a polypeptide according to claim 3, or a functional fragment thereof;
(ab) a binding target/agent of said polypeptide or functional fragment thereof; and
(ac) a candidate agent under conditions whereby said polypeptide or functional fragment thereof specifically binds to said binding target/agent at a reference affinity;
(b) detecting the binding affinity of said polypeptide or functional fragment thereof to said binding target to determine an affinity for the agent; and
(c) determining a difference between affinity for the agent and the reference affinity.

24. A method for screening for an agent, which modulates/effects the activity of a CG7042, astray, string, or CG1401 homologous polypeptide, particularly of a polypeptide according to claim 3, comprising the steps of
(a) incubating a mixture comprising
(aa) said polypeptide or a functional fragment thereof;
(ab) a candidate agent
under conditions whereby said polypeptide or functional fragment thereof has a reference activity;
(b) detecting the activity of said polypeptide or functional fragment thereof to determine an activity in presence of the agent; and
(c) determining a difference between the activity in the presence of the agent and the reference activity.

25. A method of producing a composition comprising the (poly)peptide identified by the method of claim 22 with a pharmaceutically acceptable carrier, diluent or/and additive.

26. The method of claim 25 wherein said composition is a pharmaceutical composition for preventing, alleviating or/and treating of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

27. Use of a (poly)peptide as identified by the method of claim 22 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

28. Use of a nucleic acid molecule as defined in claim 3 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

29. Use of a polypeptide as defined in claim 3 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

30. Use of a vector as defined in claim 7 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

31. Use of a host cell as defined in claim 20 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

32. Use of a CG7042, astray, string, or/and CG1401 homologous nucleic acid molecule or/and of a functional fragment thereof for the production of a non-human transgenic animal which over- or under-expresses the CG7042, astray, string, or CG1401 homologous gene product.

33. Kit comprising at least one of
(a) a CG7042, astray, string, or/and CG1401 homologous nucleic acid molecule or/and a functional fragment thereof;
(b) a CG7042, astray, string, or/and CG1401 homologous amino acid molecule or/and a functional fragment or/and an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or/and another modulator/effector of the nucleic acid of (a) or/and the polypeptide of (b), (e), or/and (f) and
(h) an anti-sense oligonucleotide of the nucleic acid of (a).
